(19) United States
(12) Patent Application Publication
(10) Pub. No.: US 2013/0017548 A1
(43) Pub. Date: Jan. 17, 2013

(54) SCREENING METHOD

(71) Applicants: Sen Takeshita, Kawasaki-shi (JP); Takashi Yamamoto, Kawasaki-shi (JP); Ayatoshi Andou, Kawasaki-shi (JP); Tomohisa Okutsu, Kawasaki-shi (JP); Agung Eviryanti, Kawasaki-shi (JP); Naoyuki Fukuchi, Kawasaki-shi (JP); Shunsuke Kageyama, Kawasaki-shi (JP)

(72) Inventors: Sen Takeshita, Kawasaki-shi (JP); Takashi Yamamoto, Kawasaki-shi (JP); Ayatoshi Andou, Kawasaki-shi (JP); Tomohisa Okutsu, Kawasaki-shi (JP); Agung Eviryanti, Kawasaki-shi (JP); Naoyuki Fukuchi, Kawasaki-shi (JP); Shunsuke Kageyama, Kawasaki-shi (JP)

(73) Assignee: AJINOMOTO, INC., Chuo-ku (JP)

(21) Appl. No.: 13/534,489

(22) Filed: Jun. 27, 2012

Related U.S. Application Data
(63) Continuation of application No. PCT/JP10/73676, filed on Dec. 28, 2010.

(30) Foreign Application Priority Data
Dec. 28, 2009 (JP) 2009-296811

(51) Int. Cl. G01N 33/573; C12Q 1/68; G01N 33/566; C12Q 1/48

(52) U.S. Cl. 435/6.12; 435/15; 435/7.8; 435/6.1; 435/7.7; 435/7.92

(57) ABSTRACT

The present invention relates to a novel method for screening for a therapeutic or prophylactic agent for an inflammatory disease.

More particularly, the present invention relates to a method for screening for a therapeutic or prophylactic agent for an inflammatory disease by selecting a substance having an inhibitory activity specific to PIKfyve by using the presence or absence of the inhibitory activity as an indicator.

### CROSS REFERENCES TO RELATED APPLICATIONS

This application is a continuation of International Patent Application No. PCT/JP2010/073676, filed on Dec. 28, 2010. The entire contents of the international application are incorporated herein by reference. The international application claims priority to Japanese Patent Application No. 2009-296811, filed on Dec. 28, 2009.

### TECHNICAL FIELD

The present invention relates to a method for screening for a therapeutic or prophylactic agent for an inflammatory disease.

### BACKGROUND ART

Interleukin-12 (IL-12) and interleukin-23 (IL-23) are cytokines that are mainly generated and secreted by phagocytes such as activated macrophage and dendritic cells, and antigen presenting cells. IL-12 is a heterodimer molecule consisting of covalently bound p35 and p40, while IL-23 is a heterodimer molecule consisting of covalently bound p19 and p40 subunit that is shared with IL-12. p35, p19 and p40 are each coded by different genes, whose productions are known to be enhanced in macrophages or the like via an increase in the gene expression due to extracellular stimulation with INF-γ or the like.

Major functions of IL-12 and IL-23 are to act on the T-cells to promote immune responses. IL-12 acts on naive T-cells and promotes differentiation into T lymphocytes of Type Th1 (type 1 helper T cells). In addition to promotion of differentiation from naive T-cells into Type Th1 T lymphocytes, IL-23 also acts on memory T-cells and IL-17-producing T-cells and results in proliferation.

Hyperfunction due to excessive production of IL-12 or IL-23 is known to be involved in worsening of pathological conditions of inflammatory diseases including various autoimmune diseases through excessive differentiation and activation of Type Th1 T cells. For example, it is known to be involved in, but not limited to, multiple sclerosis, systemic sclerosis, sepsis, myasthenia gravis, autoimmune neurological disease, Guillain-Barre syndrome, autoimmune uveitis, autoimmune hemolytic anemia, malignant anemia, autoimmune thrombocytopenia, temporal arteritis, antiphospholipid syndrome, vasculitis, Wegener's granulomatosis, Behcet's disease, psoriasis, psoriatic arthritis, herpetic dermatitis, pemphigus vulgaris, vitiligo, Crohn's disease, ulcerative colitis, interstitial pulmonary fibrosis, myelofibrosis, hepatic fibrosis, myocarditis, autoimmune thyroid disease, thyroiditis, primary biliary cirrhosis, autoimmune hepatitis, immune-mediated diabetes mellitus, Graves' disease, Hashimoto's thyroiditis, autoimmune oophoritis and orchitis, autoimmune adrenalitis, rheumatoid arthritis, juvenile rheumatoid arthritis, systemic lupus erythematosus, scleroderma, polymyositis, dermatomyositis, spondyloarthropathy, ankylosing spondylitis, Sjogren's syndrome, connective tissue disease, graft-versus-host disease and ischemic vascular disorder. Therefore, suppression of the functions of IL-12 or IL-23 is expected to ameliorate these inflammatory diseases.

So far, a number of therapeutic agents have been developed based on IL-12- and IL-23-suppressing actions of anti-IL-12 and anti-IL-23 antibodies, but they are not yet satisfactory. Although anti-IL-12/23 antibody has been found to have a therapeutic effect on psoriasis in clinical trials, there are several problems. First problem involves cost. In general, treatments using antibodies have a great burden on patients and the healthcare system in terms of cost. Second problem is the method of administration. Since treatments with antibodies require intravenous administration, burdens on patients, for example, hospital visits for every administration, is considered to be great. Hence, development of a novel inexpensive agent that can orally be administered for suppressing IL-12 or IL-23 function is considered to be necessary.

One method for suppressing IL-12 and IL-23 functions without using an antibody may be a method that suppresses IL-12 and IL-23 productions. Specifically, according to this method, a therapeutic agent is developed that acts on an intracellular target molecule in IL-12- and IL-23-producing cells such as activated macrophages, thereby suppressing IL-12 and IL-23 productions. Since this method allows development of a therapeutic agent such as a low-molecular compound that can orally be administered and that is less expensive than an antibody, it is extremely beneficial for patients.

There have been a number of reports on low-molecular compounds that suppress IL-12/IL-23 productions, but most of these compounds were weak and insufficient in terms of suppressive action. For example, steroid, 1,25-dihydroxy vitamin D3, acetylsalicylic acid (ASA), retinoid, thalidomide, prostaglandin E2, s2 agonist, phosphodiesterase inhibitor and the like have been reported. Thalidomide is likely to have the strongest suppressive action among them, but it is also reported that thalidomide cannot sufficiently suppress IL-12 and IL-23 productions strongly induced by INF-γ.

Meanwhile, a novel low-molecular compound was recently reported that showed an action of IL-12 and/or IL-23 production suppression (hereinafter, also referred to as “IL-12/23 production suppression”) (Patent document 1). This compound with an IL-12/23 production suppressive action has been reported that it sufficiently suppresses productions of IL-12 and IL-23 strongly induced by INF-γ at the cell level (Non-patent document 1), that it has been shown to exhibit an anti-inflammatory action in a model animal (Non-patent document 2), that it has been confirmed to be safe by clinical trials in human (Non-patent document 3) and the like. However, its target molecule has not been reported so far and remains unclear.

Although Non-patent document 1 reports that the compounds having an IL-12/23 production suppressive action suppress IL-12 and IL-23 genes at transcription level, it does not suggest the target molecule directly affected by these compounds. In addition, Patent document 1 reports that a transcription factor called c-Rel is involved in the action mechanism of the compound having an IL-12/23 production suppressive action, but there is no evidence of c-Rel being the directly affected target molecule, nor there is any suggestion about a target molecule.

### PRIOR ART DOCUMENTS

### Patent Document

[Patent document 1] US Patent Publication No. 2008-0227114

### Non-patent Documents

[Non-patent document 1] Blood. 2007 Feb. 1; 109(3):1156-64. Epub 2006 Oct. 19
[Non-patent document 2] Arthritis Res Ther. 2008; 10(5):R122. Epub 2008 Oct. 13
[Non-patent document 3] Inflamm Bowel Dis. 2006 July; 12(7):558-65

### SUMMARY OF THE INVENTION

### Problem to be Solved by the Invention

Under the above-described circumstances, development of a novel method for screening a therapeutic or prophylactic agent for an inflammatory disease has been desired.

### Means for Solving the Problem

The present inventors found that PIKfyve is involved in IL-12/23 production. Specifically, they found that inhibition of lipid kinase, PIKfyve, can suppress production of IL-12/23, thereby accomplishing the present invention.

More specifically:

1) based on examinations using multiple compounds, they found that the strength of IL-12- and IL-23-production inhibitory activity of mouse macrophage correlates with the strength of PIKfyve inhibitory activity;
2) they found that a compound with a strong PIKfyve inhibitory activity has an effect of suppressing intestinal inflammation in an enterocolitis model mouse; and
3) they found that gene knockdown in mouse macrophage using siRNA designed based on PIKfyve sequence (hereinafter, also referred to as “PIKfyve-siRNA”) and siRNA designed based on Vac 14 sequence, an activator of PIKfyve (hereinafter, also referred to as “Vac 114-siRNA”) results in suppression of IL-12 and IL-23 productions.

From these findings, they found out a novel method for screening a therapeutic or prophylactic agent for an inflammatory disease based on the understanding of PIKfyve functions that the presence or absence of a PIKfyve-specific inhibitory activity can be used as an indicator to select a substance having this activity.

Thus, the present invention relates to a screening method and the like below.

[1] A method for screening a prophylactic or therapeutic agent for an inflammatory disease, comprising a step of measuring PIKfyve inhibitory activity of a test substance to identify a substance having this inhibitory activity.
[2] The method according to [1] above, wherein the PIKfyve inhibitory activity is measured by using phosphorylation by ATP or labeled ATP as an indicator.
[3] The method according to either one of [1] and [2] above, wherein the PIKfyve inhibitory activity is measured by using a substrate selected from phosphatidylinositol, phosphatidylinositol 3-phosphate or a derivative thereof, or Rab9 effector p40 or a partial sequence thereof.
[4] The method according to [1] above, wherein the PIKfyve inhibitory activity is measured by using binding between PIKfyve and a probe selected from labeled ATP and PIKfyve inhibitors as an indicator.
[5] The method according to [1] above, wherein the PIKfyve inhibitory activity is caused by an inhibitory activity for Vac14.
[6] The method according to either one of [1] and [5] above, wherein the PIKfyve inhibitory activity is an inhibitory activity against binding between PIKfyve and Vac14.
[7] The method according to any one of [1], [5] and [6] above, wherein the PIKfyve inhibitory activity is measured using PIKfyve or a partial sequence thereof, or Vac14 or a partial sequence thereof as a probe.
[8] The method according to [1] above, wherein the PIKfyve inhibitory activity is measured using cellular vacuolation as an indicator.
[9] The method according to any one of [1] to [8] above, wherein the test substance is selected from a low-molecular compound, an antibody, an antisense oligonucleotide, siRNA and an aptamer.

In addition, the present invention also relates to the following pharmaceutical agent, therapeutic method and the like.

[10] The method according to [1] above, wherein the inflammatory disease is an IL-12/23-overproduction-related disease.
[11] A prophylactic or therapeutic agent for an inflammatory disease, comprising a PIKfyve inhibitor.
[12] A prophylactic or therapeutic agent for an inflammatory disease, wherein an PIKfyve inhibitor is a substance obtained by the method according to [1] above.
[13] The prophylactic or therapeutic agent according to [12] above, wherein the PIKfyve inhibitor is an antibody for PIKfyve, or an antisense oligonucleotide, siRNA or aptamer for PIKfyve gene or Vac14 gene.
[14] A prophylactic or therapeutic method for an inflammatory disease, comprising a step of administering an effective amount of a PIKfyve inhibitor to a patient.
[15] The prophylactic or therapeutic method according to [12] above, wherein the PIKfyve inhibitor is a substance obtained by the method according to [1] above.
[16] The prophylactic or therapeutic method according to either one of [12] and [13] above, wherein the PIKfyve inhibitor is an antibody, or an antisense oligonucleotide, siRNA or aptamer for PIKfyve gene or Vac14 gene.
[17] Use of a PIKfyve inhibitor for producing a prophylactic or therapeutic agent for an inflammatory disease.

### EFFECT OF THE INVENTION

A screening method of the present invention is useful for creating a novel prophylactic/therapeutic agent for an inflammatory disease.

### BRIEF DESCRIPTION OF THE DRAWINGS

FIG. 1 shows inhibitory activities of compounds against various lipid kinases.

FIG. 2 shows correlation between PIKfyve inhibitory activities and mouse peritoneal macrophage IL-12-production inhibitory activity of compounds.

FIG. 3 shows an inflammation suppressive action of a PIKfyve-inhibiting compound in a mouse enterocolitis model.

FIG. 4 shows IL-12-production suppressive actions by PIKfyve and Vac14 knockdown.

### EMBODIMENTS FOR CARRYING OUT THE INVENTION

Hereinafter, terms used herein will be described.

“PIKfyve” is one type of lipid kinases and is an enzyme having an activity of phosphorylating position 5 in phosphatidylinositol or phosphatidylinositol 3-phosphate. In addition, PIKfyve also has a protein kinase activity and phosphorylates serine residues of p40 protein, an effector for Rab9 protein. PIKfyve is called Fabi in a budding yeast and PIKfyve, PIP5K3, p235 or the like in a mammal.

Examples of the amino acid sequence of PIKfyve include, but not limited to, SEQ ID NOS:2 and 4, and Genbank Accession Nos: NP_{—}055855.1, Q9Y217 and AAR19397.

Examples of the nucleotide sequence of PIKfyve gene include, but not limited to, SEQ ID NOS:1 and 3, and Genbank Accession Nos: NM_{—}015040, AY457063 and BC032389 (cDNA).

PIKfyve comprises a homolog, a mutant, a variant and the like of PIKfyve that retains an activity of phosphorylating position 5 in phosphatidylinositol and phosphatidylinositol 3-phosphate or an ability to phosphorylate the serine residues of p40 protein.

Such a homolog, mutant or variant comprises a protein with an amino acid sequence having an identity of, for example, 80% or higher, 85% or higher, 90% or higher, 95% or higher, 97% or 99% with SEQ ID NO:2 or 4, and a protein with an amino acid sequence having, for example, 1 to 20, 1 to 15, 1 to 10, 1 to several (for example, 9), 1 to 5, 1 to 3 or 1 to 2 amino acids deleted from, substituted in, inserted into and/or added to the amino acid sequence represented by SEQ ID NO:2 or 4.

PIKfyve gene can be hybridized with sequences represented by SEQ ID NO:1 or 3, GenBank Accession No. AY457063 or BC032389 or the like under stringent conditions, and comprises DNA that retains PIKfyve activity (the activity of phosphorylating position in phosphatidylinositol or phosphatidylinositol 3-phosphate, the ability to phosphorylate the serine residues of p40 protein or the like).

A partial sequence of PIKfyve may be any sequence containing all or part of CCT domain of PIKfyve that has been reported to have at least a binding ability with Vac14, for example, a sequence containing amino acid residues 346-1134 of human PIKfyve or a part thereof. The length is preferably that of a peptide having 5 to 1000, and preferably 501 to 1000 amino acid residues.

“Vac14” is an activator protein of PIKfyve, which forms a PIKfyve-Vac14 complex by binding to PIKfyve via the HEAT-repeat motifs on the N-terminal side. This complex formation is considered to be necessary for intracellular activation of PIKfyve. Vac14 is called Vac14 in a budding yeast, and Vac14, ArPIKfyve, TAX1BP2 or the like in a mammal.

Examples of the amino acid sequence of Vac 14 include, but not limited to, the amino acid sequences represented by SEQ ID NOS:6 and 8, and Genbank Accession Nos: NP_{—}060522.3, AAI25110.1 and AAI25109.1.

Examples of the nucleotide sequence of Vac 14 gene include DNAs represented by SEQ ID NOS:5 and 7.

Vac14 comprises a homolog, a mutant, a variant and the like of Vac14 that retains the ability to form a PIKfyve-Vac14 complex.

Such a homolog, mutant or variant comprises a protein with an amino acid sequence having an identity of, for example, 80% or higher, 85% or higher, 90% or higher, 95% or higher, 97% or 99% with SEQ ID NO:6 or 8, and a protein with an amino acid sequence having, for example, 1 to 20, 1 to 15, 1 to 10, 1 to several (for example, 9), 1 to 5, 1 to 3 or 1 to 2 amino acids deleted from, substituted in, inserted into and/or added to the amino acid sequence represented by SEQ ID NO:6 or 8.

Vac14 gene can be hybridized with sequences represented by SEQ ID NO:5 or 7, GenBank Accession No. BC125109.1 or NM_{—}018052 or the like under stringent conditions, and comprises DNA that retains the ability to form a PIKfyve-Vac 4 complex.

A partial sequence of Vac14 is a sequence containing 1st to 11th HEAT-repeat motifs on the N-terminal side of Vac 14 or a part thereof that has been reported of its binding ability with PIKfyve, which is preferably a peptide of 5 to 500, or 100 to 500 amino acid residues.

p40 interacts with PIKfyve. PIKfyve interaction and subsequent PIKfyve-catalyzed p40 phosphorylation anchor p40 to discrete membranes to facilitate late endosome-to-trans-Golgi transport. Moreover, p40 can bind to Rab9-GTP and stimulate endosome-to-trans-Golgi transport (see Diaz et al., J. Cell Biol. 138: 283-90, 1997, etc.). p40 is an effector for Rab9 and also called Rab9 effector p40, or Rab9p40.

Examples of the amino acid sequence of p40 include, but not limited to, SEQ ID NO: 10, and Genbank Accession Nos: NP_{—}005824 and CAG33118. p40 comprises a homolog, a mutant, a variant and the like of p40.

Examples of the nucleotide sequence of p40 gene include, but not limited to, SEQ ID NO:9, and Genbank Accession Nos: NM_{—}015040, NM_{—}005833.2 and CR456837.1 (cDNA).

p40 comprises a homolog, a mutant, a variant and the like of PIKfyve-phosphorylated p40.

Such a homolog, mutant or variant comprises a protein with an amino acid sequence having an identity of, for example, 80% or higher, 85% or higher, 90% or higher, 95% or higher, 97% or 99% with SEQ ID NO:10, and a protein with an amino acid sequence having, for example, 1 to 20, 1 to 15, 1 to 10, 1 to several (for example, 9), 1 to 5, 1 to 3 or 1 to 2 amino acids deleted from, substituted in, inserted into and/or added to the amino acid sequence represented by SEQ ID NO: 10.

p40 gene can hybridize with the sequence represented by SEQ ID NO:9, or GenBank Accession No. NM_{—}005833.2 or CR456837.1 under stringent conditions, and comprises those that can be phosphorylated by PIKfyve.

A partial sequence of p40 is a sequence including the Ser residues contained in p40 protein, which is a peptide containing preferably 5 to 300, and more preferably 100 to 300 amino acid residues.

Here, the phrase “DNA that hybridizes under stringent conditions” refers to DNA that can be obtained by employing a colony hybridization technique, a plaque hybridization technique or a Southern hybridization technique using whole or a part of DNA containing a nucleotide sequence complementary to the nucleotide sequence represented by SEQ ID NO:1, 3, 5, 7 or 9 as a probe. A hybridization technique that can be employed may be, for example, a technique described in Molecular Cloning 3rd Ed., Current Protocols in Molecular Biology, John Wiley & Sons 1987-1997 or the like.

As used herein, the term “stringent conditions” may refer to any of lowly stringent conditions, mildly stringent conditions and highly stringent conditions. For example, “lowly stringent conditions” may be 5×SSC, 5×Denhardt solution, 0.5% SDS and 50% formamide at 32° C. Furthermore, “mildly stringent conditions” may be, for example, 5×SSC, 5×Denhardt solution, 0.5% SDS and 50% formamide at 42° C. “Highly stringent conditions” may be, for example, 5×SSC, 5×Denhardt solution, 0.5% SDS and 50% formamide at 50° C. Under these conditions, higher temperature is expected to result in efficient acquirement of a polynucleotide (for example, DNA) with higher homology. However, multiple factors are considered to influence the stringency of hybridization, including temperature, probe concentration, probe length, time, ionic strength, salt concentration and the like, which can appropriately be selected by those skilled in the art to realize similar stringency.

In a case where a commercially available kit is used for hybridization, for example, AlkphosDirect Labelling Reagents (produced by GE Healthcare Japan) can be used. In this case, in accordance with the protocol attached to the kit, incubation with a labeled probe is carried out overnight, then the membrane is washed with a primary wash buffer containing 0.1% (w/v) SDS under the conditions of 55° C., and subsequently the hybridized polynucleotide (for example, DNA) can be detected.

Other than these, examples of hybridizable DNAs include DNAs having an identity of 80% or higher, 81% or higher, 82% or higher, 83% or higher, 85% or higher, 90% or higher, 95% or higher, 97% or higher, or 99% or higher with a polynucleotide coding for the amino acid sequence represented by SEQ ID NO:2, 4, 6, 8 or 10 when calculated with a homology search software such as FASTA and BLAST using default parameters.

Identity of an amino acid sequence or a nucleotide sequence can be determined using algorithm BLAST by Karlin and Altschul (Proc. Natl. Acad. Sci. USA, 87, 2264-2268, 1990; Proc. Natl. Acad. Sci. USA, 90, 5873, 1993). Programs called BLASTN and BLASTX based on the algorithm of BLAST have been developed (Altschul S F, et al: J. Mol. Biol. 215: 403, 1990). When BLASTN is used to analyze a nucleotide sequence, parameters are, for example, score=100 and wordlength=12. When BLASTX is used to analyze an amino acid sequence, parameters are, for example, score=50 and wordlength=3. When BLAST and Gapped BLAST programs are used, default parameters for each program are used.

A “PIKfyve inhibitory activity” refers to a degree of the effect to reduce or annihilate the enzymatic activity of PIKfyve. Specifically, a PIKfyve inhibitory activity can be determined by measuring a degree of reduction in phosphorylation or binding by a test substance, namely, phosphorylation of position 5 in phosphatidylinositol or phosphatidylinositol 3-phosphate, phosphorylation of Rab9 effector p40, binding between PIKfyve and ATP or the like, caused by the enzymatic activity of PIKfyve. Measurement of a PIKfyve inhibitory activity is described, for example, in EMBO Rep. 2008 February; 9(2):164-70. Epub 2008 Jan. 11.

PIKfyve inhibitory activity resulting from Vac 14 inhibitory activity means that the enzymatic activity of PIKfyve is reduced or annihilated by inhibiting the function of Vac14. Inhibition of Vac14 function include eliminating or reducing the function by altering the structure of Vac14, or disturbing the formation of a PIKfyve-Vac 14 complex by inhibiting binding between PIKfyve and Vac14, thereby reducing or annihilating the enzymatic activity of PIKfyve. Preferably, it is inhibition of binding between PIKfyve and Vac 14.

The term “cellular vacuolation” refers to a change in cell morphology resulting from inhibition of intracellular vesicular transport due to PIKfyve inhibition or Vac 14 inhibition. By measuring the degree of cellular vacuolation, inhibitory activity of PIKfyve or inhibitory activity of Vac 14 can be determined.

Examples of the “PIKfyve inhibitor” include the above-described low-molecular compound having a PIKfyve inhibitory activity with IC₅₀ of 1 mM or less, an antibody for PIKfyve, or an antisense oligonucleotide, siRNA or aptamer for PIKfyve gene or Vac14 gene.

The “PIKfyve inhibitor” also includes a derivative of a PIKfyve inhibitor. A derivative of a PIKfyve inhibitor refers to a substance that can be obtained by derivatizing a PIKfyve inhibitor.

Derivatization refers to virtual or actual synthesis of a compound obtained by replacing a certain atom or group in a lead compound with other atom or group, or a compound obtained through addition reaction on a lead compound. For example, the lead compound may be a substance obtained by a screening method of the present invention.

Derivatization of a PIKfyve inhibitor can be carried out while considering water solubility/lipid solubility, stability, disposition, biological availability, toxicity and other properties of the resulting derivative as appropriate such that it retains inhibitory capacity for PIKfyve.

For example, derivatization of a PIKfyve inhibitor may be carried out based on SBDD (Structure-Based Drug Design) or CADD (Computer-Aided Drug Design). Examples of such design include virtual screening, de novo design, pharmacophore analysis and QSAR (Quantitative Structure Activity Relationship). If conformational information of the protein itself or the target site of the protein is necessary upon such design, conformational information known from a structural analysis technique such as NMR, X-ray crystallographic analysis or radiation analysis, if any, can be used. If conformation is unknown, information obtained by a structural prediction method such as a homology method or Threading method is used. In the case of virtual screening, a program known per se can be used, examples of such program being DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AutoDock (Morris, G. M. et al., J. Comput. Chem., 1998, 19, 1639), ICM (Abagyan, R. A. et al., J. Comput. Chem., 1994, 15, 488).

Examples of a test substance used in a method of the present invention include a low-molecular compound, a peptide, a protein, an antibody, an antisense oligonucleotide, siRNA, an aptamer and the like.

A “low-molecular compound” refers to a low-molecular organic compound, which may be produced by using a method known in the art of synthetic organic chemistry. Preferably, it is an organic compound with a molecular weight of 1000 or less. The low-molecular compound also comprises a salt, a hydrate or a solvate thereof. In addition, an available compound library or the like can also be used.

According to the present invention, a “protein” or a “peptide” may be a commercially available product. A peptide can be acquired by appropriately employing a known method such as (1) a chemical synthesis method or (2) a synthesis method using enzymatic reaction. When the number of amino acid residues contained is relatively small, i.e., 2 to 3 residues, a chemical synthesis method is convenient. In the case of chemical synthesis, it may be carried out by synthesizing or semi-synthesizing the oligopeptide using a peptide synthesizer.

An “antibody” may be either a monoclonal antibody or a polyclonal antibody for PIKfyve, Vac14 or p40. A monoclonal antibody can be prepared, for example, according to a method described in J Virology (1992); 66: 5999-6007.

An “antisense oligonucleotide” refers to nucleotides that are complementary to or that can hybridize with a sequence of 5 to 100 consecutive nucleotides in DNA of PIKfyve gene or Vac14 gene, which may be either DNA or RNA, and which may be modified as long as it does not interfere with the function. As used herein, the term “antisense oligonucleotide” not only comprises those whose nucleotides are completely complementary to the corresponding nucleotides constituting a predetermined region of DNA or mRNA, but it may also contain some mismatches as long as the DNA or mRNA can stably hybridize with the oligonucleotide.

An antisense oligonucleotide may be modified. Appropriate modification can render the antisense oligonucleotide to be less biodegradable, and more stable for inhibiting PIKfyve. Examples of such modified oligonucleotide include antisense oligonucleotides having modification types such as S-oligo type (phosphorothioate type), C-5 thiazole type, D-oligo type (phosphodiester type), M-oligo type (methylphosphonate type), peptide nucleic acid type, phosphodiester bond type, C-5 propynyl pyrimidine type, 2-O-propyl ribose, 2′-methoxyethoxyribose type and the like. Furthermore, an antisense oligonucleotide may be those having at least some of the oxygen atoms constituting the phosphate groups substituted or modified into sulfur atoms. Such an antisense oligonucleotide is particularly superior in nuclease resistance, water solubility, and affinity to RNA. An example of an antisense oligonucleotide having at least some of the oxygen atoms constituting the phosphate groups substituted or modified into sulfur atoms includes an oligonucleotide of S-oligo type.

The number of nucleotides in an antisense oligonucleotide is preferably 50 or less, and more preferably 25 or less. An excessive number of nucleotides increases the labor and the cost for oligonucleotide synthesis and also reduces the yield. At the same time, the number of nucleotides of the antisense oligonucleotide is 5 or more, and preferably 9 or more. If the number of nucleotides is 4 or less, specificity for the target gene will be reduced, which is unfavorable.

An antisense oligonucleotide (or a derivative thereof) can readily be synthesized by an ordinary method, for example, with a commercially available DNA synthesizer (e.g., AppliedBiosystems, etc.). The antisense oligonucleotide can be obtained by a synthetic method such as a solid-phase synthetic method using phosphoramidite, a solid-phase synthetic method using hydrogen phosphonate and the like.

An antisense oligonucleotide is preferably one that corresponds to at least 15 consecutive nucleotides in the nucleotide sequence represented by SEQ ID NO: 1, 3, 5 or 7. More preferably, it is an antisense oligonucleotide that contains an initiation codon in the at least 15 consecutive nucleotides described above.

“A derivative of phosphatidylinositol, phosphatidylinositol 3-phosphate” comprises phosphatidylinositol(3)monophosphate (PtdIns(3)P), phosphatidylinositol(3,4) diphosphate (PtdIns(3,4)P2) and the like.

siRNA refers to double-stranded RNA comprising a part of RNA coding for PIKfyve or Vac14 and RNA complementary thereto. Specifically, siRNA (Example) is used that includes a sense strand having the nucleotide sequence represented by SEQ ID NO:11 and an antisense strand having the nucleotide sequence represented by SEQ ID NO:12.

siRNA can be designed and produced based on a sequence of a polynucleotide of the present invention according to a known method (e.g., Nature, vol. 411, page 494, 2001).

siRNA can also be obtained by employing a software such as siRNA Target Finder [http://www.ambion.com/jp/techlib/misc/siRNA_finder.html](Ambion). For example, the following sequences may be obtained as siRNA for PIKfyve gene or Vac14 gene designed by the above-mentioned software.

PIKfyve 5′→3′ each
Sense strand siRNA:
(SEQ ID NO: 13)
GACGUCCCCAACACUGGACtt
Antisense strand siRNA:
(SEQ ID NO: 14)
GUCCAGUGUUGGGGACGUCtt
Sense strand siRNA:
(SEQ ID NO: 15)
CACUGGACUCUGCUAAUGAtt
Antisense strand siRNA:
(SEQ ID NO: 16)
UCAUUAGCAGAGUCCAGUGtt
Sense strand siRNA:
(SEQ ID NO: 17)
UGAUUUGCCUCGAUCUCCUtt
Antisense strand siRNA:
(SEQ ID NO: 18)
AGGAGAUCGAGGCAAAUCAtt
Sense strand siRNA:
(SEQ ID NO: 19)
CAGCAGCCUUUGAGUGGAAtt
Antisense strand siRNA:
(SEQ ID NO: 20)
UUCCACUCAAAGGCUGCUGtt
Sense strand siRNA:
(SEQ ID NO: 21)
AAAGCAGCUUAAUGAGGAAtt
Antisense strand siRNA:
(SEQ ID NO: 22)
UUCCUCAUUAAGCUGCUUUtt
Sense strand siRNA:
(SEQ ID NO: 23)
GGAAAGCAGAACCUACCUUtt
Antisense strand siRNA:
(SEQ ID NO: 24)
AAGGUAGGUUCUGCUUUCCtt
Sense strand siRNA:
(SEQ ID NO: 25)
CCUACCUUUGGAGGUCAUGtt
Antisense strand siRNA:
(SEQ ID NO: 26)
CAUGACCUCCAAAGGUAGGtt
Sense strand siRNA:
(SEQ ID NO: 27)
CGCCUCAAGGAAAUCAUGGtt
Antisense strand siRNA:
(SEQ ID NO: 28)
CCAUGAUUUCCUUGAGGCGtt
Sense strand siRNA:
(SEQ ID NO: 29)
GUUAUGCUCAUUCCACAGAtt
Antisense strand siRNA:
(SEQ ID NO: 30)
UCUGUGGAAUGAGCAUAACtt
Sense strand siRNA:
(SEQ ID NO: 31)
CAUAUGUUAGGACAGAGACtt
Antisense strand siRNA:
(SEQ ID NO: 32)
GUCUCUGUCCUAACAUAUGtt
Vac14 5′ →3′ each
Sense strand siRNA:
(SEQ ID NO: 33)
CCCCGAGAAGGAUUUCGCGtt
Antisense strand siRNA:
(SEQ ID NO: 34)
CGCGAAAUCCUUCUCGGGGtt
Sense strand siRNA:
(SEQ ID NO: 35)
AAGCGGAAGGUGGCAGCGCtt
Antisense strand siRNA:
(SEQ ID NO: 36)
GCGCUGCCACCUUCCGCUUtt
Sense strand siRNA:
(SEQ ID NO: 37)
AUCAAGCAUGUGAUCCAGAtt
Antisense strand siRNA:
(SEQ ID NO: 38)
UCUGGAUCACAUGCUUGAUtt
Sense strand siRNA:
(SEQ ID NO: 39)
GGAGCUGAUCGAGCCAGUGtt
Antisense strand siRNA:
(SEQ ID NO: 40)
CACUGGCUCGAUCAGCUCCtt
Sense strand siRNA:
(SEQ ID NO: 41)
GCGGAUCUGAGCUCCUAGAtt
Antisense strand siRNA:
(SEQ ID NO: 42)
UCUAGGAGCUCAGAUCCGCtt
Sense strand siRNA:
(SEQ ID NO: 43)
GUUUGACCUGGUGAGCUUCtt
Antisense strand siRNA:
(SEQ ID NO: 44)
GAAGCUCACCAGGUCAAACtt
Sense strand siRNA:
(SEQ ID NO: 45)
AUUAAGAAGAACCCCUCCAtt
Antisense strand siRNA:
(SEQ ID NO: 46)
UGGAGGGGUUCUUCUUAAUtt
Sense strand siRNA:
(SEQ ID NO: 47)
GUUUGCUGAGAUGGCCAACtt
Antisense strand siRNA:
(SEQ ID NO: 48)
GUUGGCCAUCUCAGCAAACtt
Sense strand siRNA:
(SEQ ID NO: 49)
AAGCAUCAAAGAAGUGGCCtt
Antisense strand siRNA:
(SEQ ID NO: 50)
GGCCACUUCUUUGAUGCUUtt
Sense strand siRNA:
(SEQ ID NO: 51)
GCUCCUGGAGGUCAGAGGCtt
Antisense strand siRNA:
(SEQ ID NO: 52)
GCCUCUGACCUCCAGGAGCtt

An aptamer is a 20 to 60-mer oligonucleotide (RNA/DNA) that specifically binds to a target protein, which has an ability to penetrate into a pocket (indent) of a protein to form a stable three-dimensional conformation, thereby inhibiting the function. A nucleotide sequence of an aptamer can be determined by a procedure called SELEX (Systematic Evolution of Ligands by Exponential enrichment) method.

First, sequences that bind to a target protein are selected from a group of nucleic acids having random nucleotide sequences and amplified. Subsequently, these selection and amplification are repeated to search for a sequence having high specificity.

An “inflammatory disease” refers to a condition or a disease associated with inflammation, and in particular an IL-12- or IL-23-production-meditated disease comprising an autoimmune disease. Specifically, examples include multiple sclerosis, systemic sclerosis, sepsis, myasthenia gravis, autoimmune neurological disease, Guillain-Barre syndrome, autoimmune uveitis, autoimmune hemolytic anemia, malignant anemia, autoimmune thrombocytopenia, temporal arteritis, antiphospholipid syndrome, vasculitis, Wegener's granulomatosis, Behcet's disease, psoriasis, psoriatic arthritis, herpetic dermatitis, pemphigus vulgaris, vitiligo, Crohn's disease, ulcerative colitis, interstitial pulmonary fibrosis, myelofibrosis, hepatic fibrosis, myocarditis, autoimmune thyroid disease, thyroiditis, primary biliary cirrhosis, autoimmune hepatitis, immune-mediated diabetes mellitus, Graves' disease, Hashimoto's thyroiditis, autoimmune oophoritis and orchitis, autoimmune adrenalitis, rheumatoid arthritis, juvenile rheumatoid arthritis, systemic lupus erythematosus, scleroderma, polymyositis, dermatomyositis, spondyloarthropathy, ankylosing spondylitis, Sjogren's syndrome, connective tissue disease, graft-versus-host disease, ischemic vascular disorder and Castleman's disease.

Among them, preferable examples include Crohn's disease, ulcerative colitis, multiple sclerosis, psoriasis, psoriatic arthritis, primary biliary cirrhosis, autoimmune uveitis and rheumatoid arthritis.

Here, diseases mediated by IL-12 or IL-23 production include, for example, IL-12/23-overproduction-related diseases where overproduction of IL-12 or IL-23 is involved in the pathological condition, and diseases associated with mutation of the receptor mechanism of IL-12 or IL-23. Examples of such IL-12/23-overproduction-related disease include multiple sclerosis, systemic sclerosis, sepsis, myasthenia gravis, autoimmune neurological disease, Guillain-Barre syndrome, autoimmune uveitis, autoimmune hemolytic anemia, malignant anemia, autoimmune thrombocytopenia, temporal arteritis, antiphospholipid syndrome, vasculitis, Wegener's granulomatosis, Behcet's disease, psoriasis, psoriatic arthritis, herpetic dermatitis, pemphigus vulgaris, vitiligo, Crohn's disease, ulcerative colitis, interstitial pulmonary fibrosis, myelofibrosis, hepatic fibrosis, myocarditis, autoimmune thyroid disease, primary biliary cirrhosis, autoimmune hepatitis, immune-mediated diabetes mellitus, autoimmune oophoritis and orchitis, autoimmune adrenalitis, rheumatoid arthritis, juvenile rheumatoid arthritis, systemic lupus erythematosus, scleroderma, polymyositis, dermatomyositis, spondyloarthropathy, ankylosing spondylitis, Sjogren's syndrome and graft-versus-host disease. In particular, psoriasis (Lancet. 2008 May 17; 371(9625):1665-74), Crohn's disease and ulcerative colitis (Gastroenterology. 2008 October; 135(4):1130-41. Epub 2008 Jul. 17, Am J Gastroenterol. 2008 March; 103(3):621-7. Epub 2007 Nov. 28) are favorable.

An example of a disease associated with mutation of the receptor mechanism of IL-23 includes Crohn's disease (Nat. Genet. 2008 August; 40(8):955-62. Epub 2008 Jun. 29). Clinical effects of an IL-12/23 inhibitor on psoriasis or psoriatic arthritis are reported in Lancet. 2008 May 17; 371(9625): 1665-74 and Lancet. 2009 Feb. 21; 373(9664): 633-40. Epub 2009 Feb. 11. In addition, J Invest Dermatol. 2009 February; 129(2): 355-8. Epub 2008 Sep. 18, Arthritis Rheum. 2008 December; 58(12):3705-9 and else also describe relevance between these diseases and IL-12 and/or IL-23. Furthermore, relevance with rheumatoid arthritis is reported in Ann Rheum Dis. 2008 February; 67(2):248-50. Epub 2007 Jul. 2. Relevance with Crohn's disease and ulcerative colitis is reported in Gastroenterology. 2008 October; 135(4):1130-41. Epub 2008 Jul. 17 and Am J Gastroenterol. 2008 March; 103(3):621-7. Epub 2007 Nov. 28. Relevance with multiple sclerosis is reported in Expert Rev Neurother. 2009 March; 9(3):319-21. Relevance with primary biliary cirrhosis is reported in N Engl J. Med. 2009 Jun. 11; 360(24):2544-55. Epub 2009 May 20.

Moreover, relevance with vasculitis (Hum Immunol. 2006 September; 67(9):735-40. Epub 2006 Jul. 20.), ankylosing spondylitis (Curr Opin Rheumatol. 2009 July; 21(4):318-23.), autoimmune thyroiditis (J Clin Endocrinol Metab. 2008 March; 93(3):1077-81. Epub 2007 Dec. 11.), systemic sclerosis (J Rheumatol. 2009 Nov. 16.), Behcet's disease (J Invest Dermatol. 2006 July; 126(7):1534-40. Epub 2006 Mar. 2.), immune-mediated diabetes mellitus (Nat. Genet. 2001 February; 27(2):218-21.), graft-versus-host disease (Biol Blood Marrow Transplant. 2009 December; 15(12):1571-7. Epub 2009 Oct. 1.) and Castleman's disease (Int J Hematol. 2007 April; 85(3):207-11.) is also reported in the respective documents.

Hereinafter, a screening method of the present invention will be described.

According to a screening of the present invention, PIKfyve inhibitory activity can be calculated using phosphorylation of position 5 in phosphatidylinositol or phosphatidylinositol 3-phosphate due to enzymatic activity of PIKfyve or phosphorylation of Rab9 effector p40, as an indicator. Alternatively, it can also be calculated by measuring a change in the binding amount between PIKfyve and Vac14, the binding amount between PIKfyve and ATP, cellular vacuolation or the like upon addition of a substance to be evaluated.

When phosphorylation is used as an indicator, a phosphorylation assay is employed whereas when an inhibitory activity against binding is to be assessed, measurement can be carried out by a binding assay using various probes. When cellular vacuolation is used as an indicator, it may be measured by an assay for morphological change using High Content Screening.

(1) Phosphorylation Assay

Using PIKfyve or a partial sequence of PIKfyve as an enzyme, and phosphatidylinositol or phosphatidylinositol 3-phosphate or a derivative thereof or Rab9 effector p40 or a partial sequence thereof as a substrate, the substrate is phosphorylated with PIKfyve in the presence of ATP or labeled ATP and a substance to be evaluated is added to the reaction system for reaction. Subsequently, a substance having an inhibitory activity against PIKfyve is selected by using the labeled ATP bound to the substrate or the phosphate group as an indicator, thereby accomplishing screening.

As labeled ATP, ³²P-labeled form can favorably be used. An example of ³²P-labeled ATP includes [γ-³²P] ATP (GE Healthcare Japan, Daiichi Kagaku).

An inhibitory activity can be measured according to a method in which an intracellular enzymatic activity is measured using a highly expressing cell or a culture cell of PIKfyve, a method using an in vitro activity measurement system, or the like.

For example, the method using a highly expressing cell of PIKfyve may be a method in which a human lymphocyte-derived Jurkat cell is suspended in a lysis buffer or the like by sonication and the supernatant resulting from centrifugation is used as a cell extract containing PIKfyve.

An in vitro measurement system may be a system in which human or mouse PIKfyve is expressed in an insect cell and purified, for example, by a known method using a baculovirus vector or the like, and the purified PIKfyve is used to measure the enzymatic activity.

As p40 or a partial sequence thereof, at least a part or whole sequence of the above-described sequence can be used, which is preferably a peptide containing Ser residues of p40 and a peptide containing preferably 5 to 300, and more preferably 100 to 300 amino acid residues.

Specifically, a method that uses p40 as a substrate can be realized by preparing p40 by expressing GST-p40 by a known method using an E. coli expression system, purifying the resultant by a method using Glutathione-agarose beads, and using the resultant as a substrate to perform in vitro activity measurement with purified PIKfyve. The E. coli expression system may be pET Expression System (Takara Bio) or the like while Glutathione-agarose beads may be Immobilized Glutathione (Thermo Scientific) or the like.

(2) Binding Assay

Any conventionally known method can be applied that uses PIKfyve as an enzyme for examining the presence or absence of binding or the presence or absence of disassociation between the substances. For example, a substance to be evaluated is added to the reaction system for reaction in the presence of PIKfyve, labeled ATP and a probe selected from PIKfyve inhibitors. Subsequently, a substance having an inhibitory activity against PIKfyve binding is selected through a step of judging the inhibitory activity of the substance to be evaluated against PIKfyve binding using the labeled ATP bound to PIKfyve as an indicator, thereby accomplishing screening.

A probe selected from PIKfyve inhibitors may be, for example, a low-molecular compound having a PIKfyve inhibitory activity, an antibody for PIKfyve, an antisense oligonucleotide, siRNA or aptamer for PIKfyve gene or Vac14 gene. For example, compounds indicated herein in Synthesis Examples 1-3 can be used.

Specifically, in order to screen for a compound that inhibits binding between a probe and PIKfyve, first, a cell or a cell membrane fraction containing PIKfyve is suspended in a buffer suitable for screening to prepare a PIKfyve sample. The buffer may be any buffer such as a phosphate buffer or Tris-hydrochloric buffer with pH 4-10 (preferably, pH 6-8) as long as it does not inhibit the binding between the probe and PIKfyve. In addition, for the purpose of reducing non-specific binding, a surfactant such as CHAPS, Tween-80, deoxycholate or digitonin may be added to the buffer. Moreover, for the purpose of preventing degradation of the receptor or the ligand by protease, a protease inhibitor such as leupeptin, PMSF, E-64 (Peptide Institute) or pepstatin can also be added. To 0.01 to 10 ml of the receptor solution, a constant amount (5000 to 500000 cpm) of the labeled probe is added, and at the same time 10⁻⁴ M to 10⁻¹⁰ M test compound is allowed to coexist. In order to find out the non-specific binding amount (NSB), a reaction tube added with an overly excessive amount of unlabeled probe is also prepared. The reaction is carried out at about 0 to 50° C., preferably about 4 to 37° C. for about 20 minutes to 24 hours, preferably about 30 minutes to 3 hours. After the reaction, the resultant is filtrated by glass fiber filter paper or the like, and washed with an appropriate amount of the same buffer. Subsequently, radioactivity remaining on the glass fiber filter paper is counted with a liquid scintillation counter or a γ-counter. Where the count (BO-NSB) resulting from subtracting the non-specific binding amount (NSB) from the count without any antagonistic substance (BO) is assumed to be 100%, a test compound whose specific binding amount (B-NSB) is, for example, 50% or less is selected as a candidate substance having an antagonistic inhibitory capacity.

(3) PIKfyve-Vac14 Binding Assay

Similarly, any known method can be used that uses PIKfyve or a partial sequence thereof to measure an activity to inhibit binding with Vac 14 or a partial sequence thereof. For example, where PIKfyve or a partial sequence thereof is used as a probe and Vac14 or a partial sequence is added as a probe, a substance to be evaluated is added to the reaction system for reaction. Subsequently, a substance having a PIKfyve inhibitory activity is selected through a step of judging an inhibitory activity against binding between PIKfyve and Vac 14 by employing any conventionally known method using binding between PIKfyve or a partial sequence thereof and Vac 14 or a partial sequence thereof as an indicator.

As a probe of PIKfyve or a partial sequence thereof, DNA with a chain length of at least 15 bp having at least a part or whole PIKfyve sequence (or a complementary sequence thereof) can be used. A preferable example may be a sequence of 1038 bp to 3402 bp, or a part thereof containing CCT domain of human PIKfyve gene that has been reported to have a binding ability to Vac14. Preferably, the length is that of a partial sequence having 15 to 3000, and preferably 1503 to 3000 nucleotides.

As a probe of Vac 14 or a partial sequence thereof, DNA with a chain length of at least 15 bp having at least a part or whole Vac 14 sequence (or a complementary sequence thereof) can be used. A preferable example may be a gene sequence containing 1st to 11th HEAT-repeat motifs on the N-terminal side of Vac14 gene that has been reported to have a binding ability to PIKfyve. The length of the partial sequence is preferably 15 to 1500, and preferably 300 to 1500 nucleotides.

Specifically, yeast Two-Hybrid method described in Fields, S & Song, O. (1989) Nature 340(6230):245-246 can be exemplified. For example, in order to carry out PIKfyve-Vac14 binding assay, first, a recombinant gene in which PIKfyve gene is fused to the DNA-binding domain of transcription factor Gal4 and a recombinant gene in which Vac 14 gene is fused to the transcription activated domain of Gal4 are prepared and transferred into a yeast cell for measurement carried out by a reporter assay according to yeast Two-Hybrid method. Matchmaker Gold Two-Hybrid System (Takara Bio) can be used for the yeast Two-Hybrid method.

Another exemplary method is Alpha Screen method described in Eglen R M, Reisine T, Roby P, et al, Curr Chem. Genom. 2008; 1:2-10. Specifically, PIKfyve-Vac14-binding assay may be Alpha screen method in which PIKfyve protein tagged with biotin and Vac14 protein tagged with GST tag sequence are prepared and mixed with streptavidin-bound donor beads and Anti-GST IgG-bound acceptor beads to perform a chemically amplified luminescence proximity homogeneous assay. The donor beads and the acceptor beads may be Streptavidin-coated Donor Beads (Perkin Elmer) and Anti-GST IgG conjugated Acceptor Beads (Perkin Elmer), respectively.

(4) Assay for Change in Cell Morphology

A cell expressing PIKfyve is used to apply any conventionally known method for examining the presence or absence of change in cell morphology. A specific example includes a method reported in Nat Cell Biol. 2010 August; 12(8):747-57.

For example, a substance to be evaluated is added to a reaction system containing a PIKfyve-expressing cell. Subsequently, a substance having a PIKfyve inhibitory activity is selected through a step of measuring the change in cell morphology using High Content Screening, thereby accomplishing screening.

A “prophylactic or therapeutic agent” according to the present invention refers to a substance obtained by the above-defined screening method of the present invention, namely, a pharmaceutical composition comprising any of a “low-molecular compound”, an “antibody”, an “antisense oligonucleotide”, “siRNA” or an “aptamer” as an effective component together with a pharmaceutically acceptable carrier.

These pharmaceutical compositions of the present invention are useful as therapeutic agents for the above-described inflammatory diseases.

Here, examples of the “pharmaceutically acceptable carrier” include an excipient, a diluent, a filler, a disintegrant, a stabilizer, a preserving agent, a buffer, an emulsifier, an aromatic agent, a coloring agent, a sweetening agent, a thickening agent, a flavoring agent, a solubilizing aid or other additives. By using one or more of such carriers, a pharmaceutical composition in a dosage form of a tablet, pill, powdered agent, granules, an injectable agent, a liquid agent, a capsule, elixir, lozenge, suspension, emulsion, syrup or the like can be prepared. These pharmaceutical compositions can be administered orally or parenterally. Other dosage forms for parenteral administration include a liquid agent for external application, suppository for enteric administration and pessary formulated by ordinary methods, which contain one or more active substances.

Although a dosage amount differs according to the age, sex, weight and condition of the patient, therapeutic effect, administration method, treating time and the type of an active component (the above-described polypeptide, antibody or the like) contained in the pharmaceutical composition, a single dosage can generally be in a range of 10 μg to 1000 mg (alternatively, 10 μg to 500 mg) per adult. However, since a dosage amount may vary according to various conditions, a dosage amount may be sufficient in an amount less than the above-mentioned dosage amount, or a dosage amount required may exceed the above-mentioned range.

Particularly in the case of an injectable agent, it may be produced by dissolving or suspending the effective component in a non-toxic pharmaceutically acceptable carrier such as physiological saline or a commercially available injectable distilled water to a concentration of 0.1 μg to 10 mg/ml carrier. The thus-produced injectable agent can be administered, once or several times a day, for a single dosage of 1 μg to 100 mg and preferably 50 μg to 50 mg per kg weight of a human patient who needs treatment. Exemplary administration forms include medically appropriate administration forms such as intravenous injection, subcutaneous injection, intradermal injection, intramuscular injection or intraperitoneal injection. Preferably, it is intravenous injection.

In some cases, an injectable agent can also be prepared as a non-aqueous diluent (for example, propylene glycol, polyethylene glycol, a vegetable oil such as olive oil, and alcohol such as ethanol), suspension or emulsion.

Sterilization of such an injectable agent can be carried out by filtration sterilization by passing through a bacteria-retaining filter, by blending a disinfectant or by irradiation. An injectable agent can be produced in a form that can be prepared before use. Specifically, it can be made into a sterile solid composition by lyophilization or the like, which can be dissolved in sterile injectable distilled water or other solvent for use.

### EXAMPLES

Hereinafter, the present invention will specifically be described by means of examples. The scope of the present invention is not limited to these examples.

### Synthesis Example 1

### Synthesis of Compound 1: (N-(3-METHYL-BENZYLIDENE)-N′-(7-MORPHOLINE-4-YL-2-PYRIDINE-4-YL-PYRAZOLO[1,5-A]PYRIMIDINE-5-YL)-HYDRAZINE)

### (Step1) 5-PYRIDINE-4-YL-2H-PYRAZOLE-3-YL-AMINE

To a THF (10 mL) solution containing 18-crown-6 (875 mg, 3.31 mmol) and tert-butoxy potassium (1M THF solution, 36.4 mL, 36.4 mmol), acetonitrile (2.09 mL, 39.7 mmol) was added at 60° C. and agitated for 5 minutes. To this solution, ethyl isonicotinate (5.00 g, 33.1 mmol) was added and agitated for 30 minutes. This suspension was filtrated and the solid was washed with diethyl ether.

The resulting solid was suspended in ethanol (60 mL), added with concentrated hydrochloric acid in ice (3.0 mL) and methyl carbazate (3.61 g, 39.7 mmol), and agitated at room temperature for 20 hours. After agitation, potassium carbonate (2.74 g, 19.8 mmol) was added to this suspension and agitated at 90° C. for an hour. The reaction solution was vacuum-concentrated, diluted with water, and subjected to extraction with ethyl acetate. The extracts were combined and dried with anhydrous sodium sulfate, and then the solvent was distilled away. The resulting solid was washed thoroughly with diethyl ether, thereby obtaining the title compound (1.33 g, 25%).

¹H-NMR (300 MHz, DMSO): δ 4.65-5.16 (br, 2H), 5.81 (br, 1H), 7.59-7.61 (m, 2H), 8.50-8.52 (m, 2H), 11.80 (br, 1H); MS (ESI) m/z 161 (M+H)⁺.

### (Step2) 5,7-DICHLORO-4-YL-2-PYRIDINE-4-YL-PYRAZOLO[1,5-A]PYRIMIDINE

To an ethanol solution (20 mL) containing 5-pyridine-4-yl-2H-pyrazole-3-yl-amine (1.29 g, 8.05 mmol), sodium ethoxide (1.21 g, 17.7 mmol) and diethyl malonate (1.34 mL, 8.86 mmol) were added and agitated for 9 hours while heating under reflux. Subsequent to agitation, the reaction solution was filtrated and the solid was washed with diethyl ether.

Phosphoryl chloride in ice (15 mL) was added to the resulting solid, and the suspension was agitated for 4 hours while heating under reflux. Phosphoryl chloride was distilled away from this reaction solution, ethanol in ice was added to the residue and agitated for 15 minutes. After concentrating the reaction solution, the residue was purified by silica-gel column chromatography (methanol/methylene chloride=1:50 to 1:20), thereby obtaining the title compound (380 mg, 18%).

¹H-NMR (300 MHz, DMSO): δ 7.79 (s, 1H), 7.84 (s, 1H), 8.41 (d, 2H, J=6.6 Hz), 8.97 (d, 2H, J=6.6 Hz); MS (ESI) m/z 265 (M+H)⁺.

### (Step3) 5-CHLORO-7-MORPHOLINE-4-YL-2-PYRIDINE-4-YL-PYRAZOLO[1,5-A]PYRIMIDINE

5,7-dichloro-4-yl-2-pyridine-4-ylpyrazolo[1,5-a]pyrimidine (380 mg, 1.43 mmol) was dissolved in 1,4-dioxane (3 mL), added with morpholine (249 μL, 2.86 mmol) and agitated at room temperature for an hour. After reaction, the suspension was concentrated, and the residue was diluted with water and subjected to extraction with ethyl acetate. The extracts were combined and dried with anhydrous sodium sulfate, and then the solvent was distilled away. The resulting solid was washed with diethyl ether, thereby obtaining the title compound (306 mg, 68%).

¹H-NMR (300 MHz, DMSO): δ 3.83-3.85 (m, 4H), 3.89-3.91 (m, 4H), 6.50 (s, 1H), 7.35 (s, 1H), 8.21 (d, 2H, J=6.2 Hz), 8.81 (d, 2H, J=6.2 Hz); MS (ESI) m/z 316 (M+H)⁺.

### (Step 4) N-(3-METHYL-BENZYLIDENE)-N′-(7-MORPHOLINE-4-YL-2-PYRIDINE-4-YL-PYRAZOLO[1,5-A]PYRIMIDINE-5-YL)-HYDRAZINE (APY0201)

5-chloro-7-morpholine-4-yl-2-pyridine-4-yl-pyrazolo[1,5-a]pyrimidine (84.0 mg, 0.266 mmol) was suspended in 1,4-dioxane (2 mL), added with hydrazine monohydrate (129 μL, 2.66 mmol) and agitated for 10 hours while heating under reflux. After agitation, the reaction solution was diluted with water and subjected to extraction with ethyl acetate. The extracts were combined and dried with anhydrous sodium sulfate, and then the solvent was distilled away.

The resulting residue was suspended in ethanol (2 mL), added with acetic acid (5.0 μL, 0.087 mmol) and 3-methylbenzaldehyde (31.3 μL, 0.266 mmol), and agitated at room temperature for an hour. After agitation, the suspension was filtrated and the resulting solid was purified by reversed-phase HPLC followed by desalination, thereby obtaining the title compound (36.2 mg, 2-step yield 33%).

¹H-NMR (300 MHz, DMSO): δ2.35 (s, 3H), 3.71-7.75 (m, 4H), 3.87-3.90 (m, 4H), 6.36 (s, 1H), 6.74 (s, 1H), 7.18 (d, 1H, J=7.6 Hz), 7.32 (t, 1H, J=7.6 Hz), 7.48 (s, 1H), 7.53 (d, 1H, J=7.6 Hz), 7.90 (d, 2H, J=4.8 Hz), 8.04 (s, 1H), 8.64 (d, 2H, J=4.8 Hz), 11.27 (s, 1H); MS (ESI) m/z 414 (M+H)⁺.

### Synthesis Example 2

### Synthesis of Compound 2: N-(1H-INDOLE-6-YLMETHYLENE)-N′-(7-MORPHOLINE-4-YL-2-PYRIDINE-4-YL-PYRAZOLO[1,5-A]PYRIMIDINE-5-YL)-HYDRAZINE

5-chloro-7-morpholine-4-yl-2-pyridine-4-ylpyrazolo[1,5-a]pyrimidine (70 mg, 0.22 mmol) was suspended in ethanol (3.0 mL), added with acetic acid (6.3 μL, 0.11 mmol) and 1H-indole-6-carbaldehyde (35 mg, 0.24 mmol), and agitated at room temperature for 16 hours. This reaction solution was filtrated and washed with ethanol and then with diethyl ether, thereby obtaining the title compound (45 mg, yield 10%). ¹H-NMR (300 MHz, DMSO-d₆); δ3.48 (bs, 4H), 3.91 (bs, 4H), 6.40 (s, 1H), 6.47 (s, 1H), 6.73 (s, 1H), 7.42-7.50 (m, 2H), 7.58-7.64 (m, 2H), 7.92 (d, 2H, J=6 Hz), 8.17 (s, 1H), 8.66 (d, 2H, J=6.3 Hz), 11.1 (s, 1H), 11.3 (s, 1H); MS (ESI) m/z 439 (M+H)⁺.

### Synthesis Example 3

### Synthesis of Compound 3: N-(3-METHYL-BENZYLIDENE)-N′-(7-MORPHOLINE-4-YL-PYRAZOLO[1,5-A]PYRIMIDINE-5-YL)-HYDRAZINE (STEP 1) SYNTHESIS OF 5-CHLORO-7-MORPHOLINE-4-YL-PYRAZOLO[1,5-A]PYRIMIDINE

5,7-dichloro-pyrazolo[1,5-a]pyrimidine (400 mg, 2.13 mmol) was dissolved in 1,4-dioxane (8 mL), added with morpholine (372 μL, 4.26 mmol) and agitated at room temperature for 30 minutes. The solvent was distilled away from this reaction mixture. The residue was diluted with water and subjected to extraction with methylene chloride. The extracts were combined and dried with anhydrous sodium sulfate, and then the solvent was distilled away, thereby obtaining the title compound (461 mg, 91%).

¹H-NMR (300 MHz, CDCl₃): δ 3.77-3.80 (m, 4H), 3.93-3.96 (m, 4H), 6.07 (s, 1H), 6.50 (d, 1H, J=2.2 Hz), 8.02 (d, 1H, J=2.2 Hz); MS (ESI) m/z 239 (M+H)⁺.

### (Step 2) SYNTHESIS OF N-(3-METHYL-BENZYLIDENE)-N′-(7-MORPHOLINE-4-YL-PYRAZOLO[1,5-A]PYRIMIDINE-5-YL)-HYDRAZINE

5-chloro-7-morpholine-4-yl-pyrazolo[1,5-a]pyrimidine (52.0 mg, 0.218 mmol) was suspended in ethanol (2 mL) and added with potassium carbonate (33.1 mg, 0.240 mmol) and hydrazine monohydrate (53.0 μL, 1.09 mmol). This suspension was agitated at 150° C. under microwave radiation for 15 minutes. This reaction solution was diluted with saturated saline and subjected to extraction with ethyl acetate. The extracts were combined and dried with anhydrous sodium sulfate, and then the solvent was distilled away. The resulting residue was suspended in ethanol (2 mL), added with acetic acid (5.0 μL, 0.087 mmol) and 3-methyl-benzaldehyde (23.2 μL, 0.197 mmol), and agitated at room temperature for an hour. This reaction mixture was filtrated and the resulting solid was washed with methanol, thereby obtaining the title compound (35.1 mg, 2-Step yield 48%).

¹H-NMR (300 MHz, DMSO): δ 2.34 (s, 3H), 3.64-3.67 (m, 4H), 3.80-3.83 (m, 4H), 6.05 (d, 1H, J=2.1 Hz), 6.29 (s, 1H), 7.16 (m, 1H), 7.30 (m, 1H), 7.46 (m, 1H), 7.51 (m, 1H), 7.88 (d, 1H, J=2.1 Hz), 8.01 (s, 1H), 11.14 (s, 1H); MS (ESI) m/z 337 (M+H)⁺.

### Laboratory Test Example 1

### Assessment of Lipid Kinase Inhibitory Activity

Lipid kinase inhibitory activity was assessed according to the method described in Biochemistry 2007, 46, 350-358. Jurkat cell derived from human lymphocyte was suspended in a lysis buffer (20 mM Hepes, 150 mM NaCl, 0.1% TritonX-100, 20 mM MnCl₂) by sonication, and the supernatant resulting from centrifugation at 100,000×g for 60 minutes was used as a cell extract. To the cell extract (5 mg/mL), a test compound (30 nM or 300 nM) or a solvent alone was added and incubated for 5 minutes. Then, an ATP derivative labeled with biotinyl acyl phosphate (20 μM) was added and incubated for 5 minutes, and the resultant was further subjected to digestion with trypsin. After adding streptavidin agarose beads, streptavidin agarose bound to biotin-labeled peptide was separated by centrifugation procedure, washed, then dissolved in a solution containing 50% CH3CN and 0.1% TFA, and incubated at 65° C. to elute the biotin-labeled peptide. The biotin-labeled peptide was analyzed by LC-MS/MS for sequence identification and measurement of the elution amount of the lipid-kinase-derived peptide so as to assess competitive inhibitory activity of the test compound for the binding of the ATP derivative to various lipid kinases. Assuming that the elution amount of the labeled peptide without addition of the test compound is 100%, the amount of reduction in the elution amount of the labeled peptide added with the test compound is indicated as inhibitory activity (%).

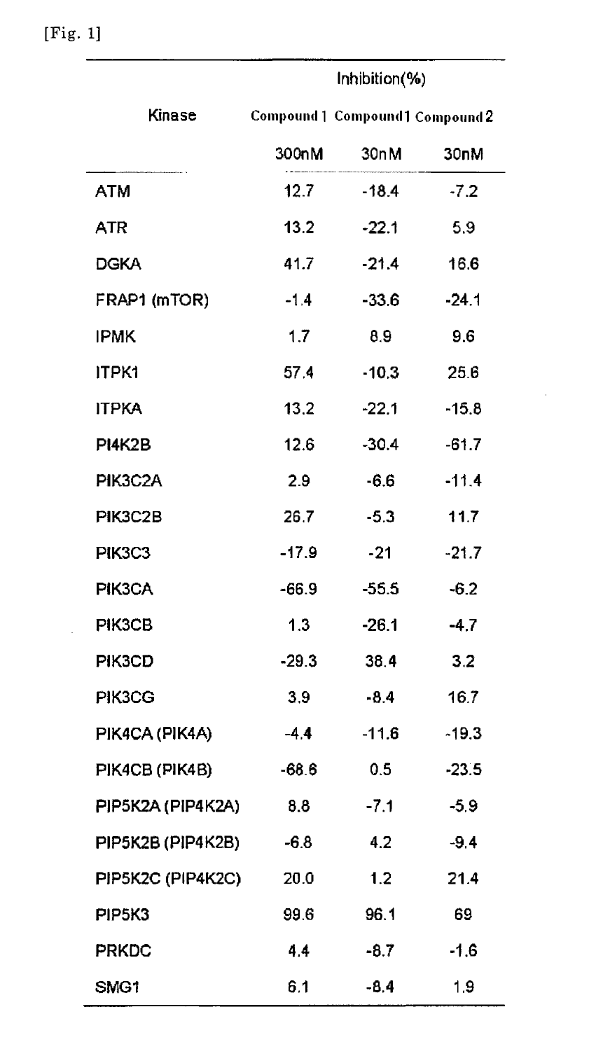

Using this assessment system, the inhibitory activity of the test compound for other lipid kinases was assessed (other lipid kinases may be commercially available or may be produced according to a method described in a known document) and the results thereof are shown in FIG. 1. Compounds 1 and 2 showed inhibitory activity selective to PIKfyve.

### Laboratory Test Example 2

### Assessment of Cytokine Suppressive Action Using Mouse Peritoneal Macrophage

Cytokine suppressive action was assessed using mouse peritoneal macrophage according to the following method. To a male Balb/c mouse (Charles River Laboratories Japan), 1 mL of 3% (w/v) thioglycollate solution was intraperitoneally administered. After 5 or 6 days, the peritoneal cell was collected and the adherent cell was used for assessment. 100 ng/mL mouse interferon-γ, 0.05% (v/v) Staphylococcus aureus Cowan I strain-derived killed culture (SAC) and the compound were added and cultured overnight. After cultivation, the survival rate was determined, and IL-12p70 (complex of IL-12p35 and p40) and TNF-α were quantitated by ELISA (BD OptEIA™ Mouse ELISA Set, BD Biosciences) using the collected culture supernatant to calculate 50% production inhibition concentration (IC₅₀).

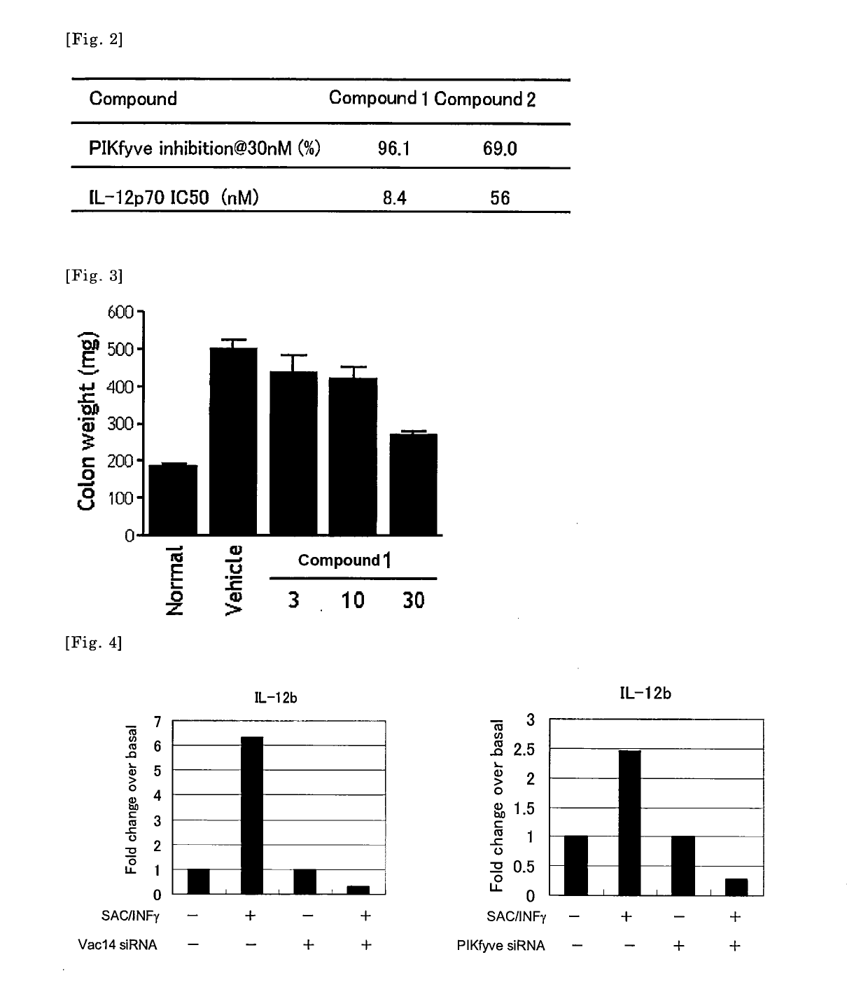

Results from the measurement of cytokine suppressive action of the test compound using this assessment system and results from the measurement of inhibitory activity for PIKfyve are shown in FIG. 2. The strength of the PIKfyve inhibitory activity of the compound showed correlation with the strength of the mouse peritoneal macrophage IL-12-production inhibitory activity.

### Laboratory Test Example 3

### Assessment of Inflammation Suppressive Action of Compound 1 in Mouse Enterocolitis Model

A method for producing a mouse IL-10^{-/-} cell transfer enterocolitis model is described, for example, in Gastroenterology. 2009 February; 136(2):564-74.e2. Epub 2008 Oct. 7.

TABLE 1

| | | | | Number of |
|---|---|---|---|---|
| Group No. | Animal | Drug | Dose | animals |
| 1 | Normal | — | — | 8 |
| 2 | Enterocolitis model | Vehicle | — | 8 |
| 3 | Enterocolitis model | Compound 1 | 3 mg/kg | 8 |
| 4 | Enterocolitis model | Compound 1 | 10 mg/kg | 8 |
| 5 | Enterocolitis model | Compound 1 | 30 mg/kg | 8 |

Measurement of an intestine weight as a drug efficacy indicator was carried out according to the following method. On the last day for autopsy, large intestine from the anus to immediately before the cecum was removed. The content of the intestine was washed with physiological saline, water was roughly removed and then the weight was measured.

Results from the assessment of the inflammation suppressive action of Compound 1 in mouse enterocolitis model using this assessment system are shown in FIG. 3. The weight of the large intestine increased by IL-10^{-/-} cell transfer but was suppressed dependent on the dosage of Compound 1. Significant suppressive action was shown in the administration group of 30 mg/kg, once a day.

### Laboratory Test Example 4

### Assessment of IL-12-Production Suppressive Action by PIKFYVE and VAC14 Knockdown

Assessment of the effects of knockdown of PIKfyve and Vac14 expressed in mouse peritoneal macrophage using siRNA method was carried out as described below. Mouse peritoneal macrophage was isolated according to the method described in Laboratory Test Example 2, and subsequently suspended in Nucleofection solution attached to Mouse macrophage nucleofection kit (Lonza) to give 3.3×10⁶ cells/mL. siRNA was added to the cell suspension to a final concentration of 1 μM and subjected to Nucleofection according to the attached protocol. The following siRNAs were used. For PIKfyve knockdown, ON-TARGETplus Non-targeting siRNA #1 (Dharmacon, D-001810-01-05) as negative control siRNA and ON-TARGETplus SMARTpool (Dharmacon, L-040127-00-0005) as PIKfyve siRNA were used. For Vac14 knockdown, Silencer Select Negative Control#1 siRNA (Ambion, 4390843) as negative control siRNA and Silencer Select Pre-designed siRNA (Sense strand: GAUUUACUCCAACAACCAAtt (SEQ ID NO: 11), Antisense strand: UUGGUUGUUGGAGUAAAUCct (SEQ ID NO:12), Ambion, s107929) as Vac14 siRNA were used.

A FBS-free RPMI1640 medium was used for culture at 37° C. in the presence of 5% CO₂ for 2 hours and then the medium was replaced by 0.5 ml of Dulbecco's MEM medium containing RPMI 1640 medium supplemented with 3% FBS for culture at 37° C. in the presence of 5% CO₂ for 4 hours. Subsequently, 3.3 ng/mL mouse interferon-γ and 0.0017% (v/v) SAC were added and cultured for 3.5 hours and then the expression level of cytokine mRNA in the cell was measured.

Extraction and purification of total RNA from the mouse peritoneal macrophage was carried out using RNeasy midi kit (Qiagen 75144) according to the attached protocol. The concentration of the purified total RNA was measured using Biophotometer (Biorad) and then capillary electrophoresis using RNA nano 6000 (Agilent Technologies) was carried out with Bioanalyzer (Agilent Technologies) to confirm the quality of the total RNA. Using the total RNA as a template and 0.5 μg of oligo dt 20-mer primer (Invitrogen), cDNA was synthesized with Superscript III (Invitrogen). cDNA synthesis was performed according to the attached protocol. At the end of the reaction, incubation was carried out with RNaseH (Invitrogen) at 37° C. for 20 minutes. The resultant removed of the remaining RNA was used as cDNA for quantitative PCR.

Quantitative PCR was carried out using SYBR Green Realtime PCR Master Mix (TOYOBO) according to the method described in the attached protocol in a 15 μl/well reaction system. cDNA was added as a template in an amount of 1/25 of the total amount of the reaction solution. Measurement was carried out with ABI7700 (Applied Biosystems) in the presence of 250 nM primer shown in Table 2. The relative expression level was calculated from the resulting Ct value. A value was calculated by dividing the relative expression level with the relative expression level of GAPDH used as the housekeeping gene. For each knockdown condition, the relative expression level normalized to GAPDH was divided by the value of the cell that had not been added with mouse interferon-a and SAC stimulation to calculate the rates of rising in IL-12b expression (fold-change over basal) caused by mouse interferon-a and SAC stimulation, which were used for verifying the effects by knockdown.

TABLE 2

| Primer | Forward | Reverse |
|---|---|---|
| GAPDH | ATCACTGCCACCCAGAAGAC | GGATGCAGGGATGATGTTCT |
| | (SEQ ID NO: 53) | (SEQ ID NO: 54) |
| IL12b | CAGCACCAGCTTCTTCATCA | TACTCCCAGCTGACCTCCAC |
| | (SEQ ID NO: 55) | (SEQ ID NO: 56) |

The results obtained by assessing the effect of PIKfyve or Vac14 knockdown on IL-12 production using the present assessment system are shown in FIG. 4. Since IL-12b expression was significantly suppressed by PIKfyve or Vac14 knockdown, IL-12-production suppression was shown to result from suppression of the expressions of these genes.

### INDUSTRIAL APPLICABILITY

A screening method of the present invention is useful for creating a novel prophylactic/therapeutic agent for an inflammatory disease.

1: A method for screening a prophylactic or therapeutic agent for an inflammatory disease, comprising:
measuring PIKfyve inhibitory activity of a test substance to identify a substance having the PIKfyve inhibitory activity.

2: The method according to claim 1, wherein the PIKfyve inhibitory activity is measured by using phosphorylation by ATP or labeled ATP as an indicator.

3: The method according to claim 1, wherein the PIKfyve inhibitory activity is measured by using a substrate selected from the group consisting of phosphatidylinositol, phosphatidylinositol 3-phosphate, a derivative of phosphatidylinositol, a derivative of phosphatidylinositol 3-phosphate, Rab9 effector p40, and a partial sequence of Rab9 effector p40.

4: The method according to claim 1, wherein the PIKfyve inhibitory activity is measured by using binding between PIKfyve and a probe selected from the group consisting of labeled ATP and PIKfyve inhibitors as an indicator.

5: The method according to claim 1, wherein the PIKfyve inhibitory activity is caused by an inhibitory activity for Vac14.

6: The method according to claim 1, wherein the PIKfyve inhibitory activity is an inhibitory activity against binding between PIKfyve and Vac14.

7: The method according to claim 1, wherein the PIKfyve inhibitory activity is measured using one of PIKfyve, a partial sequence of PIKfyve, Vac14, and a partial sequence of Vac14 as a probe.

8: The method according to claim 1, wherein the PIKfyve inhibitory activity is measured using cellular vacuolation as an indicator.

9: The method according to claim 1, wherein the test substance is selected from the group consisting of a low-molecular compound, an antibody, an antisense oligonucleotide, siRNA and an aptamer.

10: The method according to claim 1, wherein the inflammatory disease is an IL-12/23-overproduction-related disease.

11: The method according to claim 2, wherein the PIKfyve inhibitory activity is measured by using a substrate selected from the group consisting of phosphatidylinositol, phosphatidylinositol 3-phosphate, a derivative of phosphatidylinositol, a derivative of phosphatidylinositol 3-phosphate, Rab9 effector p40, and a partial sequence of Rab9 effector p40.

12: The method according to claim 5, wherein the PIKfyve inhibitory activity is an inhibitory activity against binding between PIKfyve and Vac14.

13: The method according to claim 5, wherein the PIKfyve inhibitory activity is measured using one of PIKfyve, a partial sequence of PIKfyve, Vac14, and a partial sequence of Vac14 as a probe.

14: The method according to claim 6, wherein the PIKfyve inhibitory activity is measured using one of PIKfyve, a partial sequence of PIKfyve, Vac14, and a partial sequence of Vac14 as a probe.


SEQUENCE LISTING (56 sequences)

SEQ ID NO: 1 - DNA, 6297 bp, Homo sapiens
  CDS (1)..(6297)
atggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaagagtatttgatttctgacactggaggacaacagctctcaataagtgacgctttcatcaaagaatccttatttaatcgccgagtagaggaaaaatccaaagagctgcctttcacacctttgggctggcatcataacaacctggagctcctgagggaggagaatggggagaaacaagccatggagaggttgctttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaaatcccaggtggaaagaagtttgattctgtggttgtcaatggctttgtttgtaccaagaacattgcacataaaaagatgaattcttgtattaaaaaccctaaaattcttctgttgaagtgttccattgagtatctctacagagaagaaactaagtttacttgcattgatcctattgtgcttcaggaaagggaattcttgaagaattatgtccagcgaatagttgatgttcgacccaccttggttcttgttgagaaaacagtgtctcggattgcccaggacatgttattggaacatggcattactttggtcattaatgtaaagtcacaagttttggaacgaatcagtcgaatgacccaaggtgatttagtgatgtcaatggaccagctgcttacgaaaccacacctgggcacttgtcacaaattttatatgcagatatttcagttgcctaatgaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaatcgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagctgccagagagtttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggcaaatcaggaagcaaaaatgagggtgatgaagagagagggcttattctgagtgatgctgtgtggtcaacaaaggtggactgtctgaatcccattaatcaccagagactttgtgtgctcttcagcagctcttctgcccagtccagcaatgctcctagtgcctgtgtcagtccttggattgtaacaatggaattttatggaaagaatgatcttacattaggaatatttttagagagatactgtttcaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcagttattctcccattcggcttcttgaagtatgtgttccactccccaaaatattcattaagcgtcaggccccattaaaagtgtcccttcttcaggatctgaaggacttctttcaaaaagtttcacaggtatatgttgccattgatgaaagacttgcatctttgaaaactgatacatttagtaaaacaagagaggaaaaaatggaagatatttttgcacagaaagagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggttgcaggaccttttccaacaggaaaagggtagaaagagaccttcagttcctccaagtcctggaagactgagacaaggggaagaaagcaagataagtgcgatggatgcatctccacggaatatttctccaggacttcagaatggagaaaaagaggatcgcttcttaacaactttgtccagccagagctccaccagttctactcatctccaattgcctacgccacctgaagtcatgtctgaacagtcagtgggagggccccctgagctagatacagccagcagttccgaagatgtgtttgatgggcatttgctgggatccacagacagccaagtgaaggaaaagtcaaccatgaaagccatctttgcaaatttgcttccaggaaatagctataatcctattccatttccttttgatccagataaacactacttaatgtatgaacatgaacgagtgcccattgcagtctgcgagaaggaacccagctccatcattgcttttgctctcagttgtaaagaataccgaaatgccttagaggaattgtctaaagcgactcagtggaacagtgccgaagaagggcttccaacaaatagtacttcagatagcagaccaaagagtagcagccctatcagattacctgaaatgagtggaggacagacaaatcgtacaacagaaacagaaccacaaccaaccaaaaaggcttctggaatgttgtccttcttcagagggacagcagggaaaagccccgatctctcttcccagaagagagagaccttacgtggagcagatagtgcttactaccaggttgggcagacgggcaaggaggggaccgagaatcaaggcgttgagcctcaagatgaagtagatggaggagatacacaaaagaagcaactcataaatcctcatgtggaacttcaattttcagatgctaatgccaagttttactgtcggctctactatgcgggagagtttcataagatgcgtgaagtgattctggacagcagtgaagaagatttcattcgttccctctcccactcatcaccctggcaggcccggggaggcaaatcaggagctgccttctatgcaactgaggatgatagatttattttgaagcaaatgcctcgtctggaagtccagtccttcctcgactttgcaccacattacttcaattatattacaaatgctgttcaacaaaagaggcccacggcgttggccaaaattcttggagtttacagaattggttataagaactctcagaacaacactgagaagaagttagatctccttgtcatggaaaatcttttctacgggagaaagatggcacaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattatagattatattcgaacatttacatgggacaaaaagcttgagatggttgtgaaatcaacaggaattttaggtggacaaggtaaaatgccaacagtggtgtctccggagttgtacaggactaggttttgtgaggcaatggacaagtatttcctaatggtaccagaccactggacaggcttgggtctgaattgctga

SEQ ID NO: 2 - protein, 2098 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALDTRRKAEPTFGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLANSASPSKRTSVSSFQSTVDSDSAASISLNVELDNVNFHIKKPSKYPHVPPHPADQKEYLISDTGGQQLSISDAFIKESLFNRRVEEKSKELPFTPLGWHHNNLELLREENGEKQAMERLLSANHNHMMALLQQLLHSDSLSSSWRDIIVSLVCQVVQTVRPDVKNQDDDMDIRQFVHIKKIPGGKKFDSVVVNGFVCTKNIAHKKMNSCIKNPKILLLKCSIEYLYREETKFTCIDPIVLQEREFLKNYVQRIVDVRPTLVLVEKTVSRIAQDMLLEHGITLVINVKSQVLERISRMTQGDLVMSMDQLLTKPHLGTCHKFYMQIFQLPNEQTKTLMFFEGCPQHLGCTIKLRGGSDYELARVKEILIFMICVAYHSQLEISFLMDEFAMPPTLMQNPSFHSLIEGRGHEGAVQEQYGGGSIPWDPDIPPESLPCDDSSLLESRIVFEKGEQENKNLPQAVASVKHQEHSTTACPAGLPCAFFAPVPESLLPLPVDDQQDALGSELPESLQQTVVLQDPKSQIRAFRDPLQDDTGLYVTEEVTSSEDKRKTYSLAFKQELKDVILCISPVITFREPFLLTEKGMRCSTRDYFAEQVYWSPLLNKEFKEMENRRKKQLLRDLSGLQGMNGSIQAKSIQVLPSHELVSTRIAEHLGDSQSLGRMLADYRARGGRIQPKNSDPFAHSKDASSTSSGKSGSKNEGDEERGLILSDAVWSTKVDCLNPINHQRLCVLFSSSSAQSSNAPSACVSPWIVTMEFYGKNDLTLGIFLERYCFRPSYQCPSMFCDTPMVHHIRRFVHGQGCVQIILKELDSPVPGYQHTILTYSWCRICKQVTPVVALSNESWSMSFAKYLELRFYGHQYTRRANAEPCGHSIHHDYHQYFSYNQMVASFSYSPIRLLEVCVPLPKIFIKRQAPLKVSLLQDLKDFFQKVSQVYVAIDERLASLKTDTFSKTREEKMEDIFAQKEMEEGEFKNWIEKMQARLMSSSVDTPQQLQSVFESLIAKKQSLCEVLQAWNNRLQDLFQQEKGRKRPSVPPSPGRLRQGEESKISAMDASPRNISPGLQNGEKEDRFLTTLSSQSSTSSTHLQLPTPPEVMSEQSVGGPPELDTASSSEDVFDGHLLGSTDSQVKEKSTMKAIFANLLPGNSYNPIPFPFDPDKHYLMYEHERVPIAVCEKEPSSIIAFALSCKEYRNALEELSKATQWNSAEEGLPTNSTSDSRPKSSSPIRLPEMSGGQTNRTTETEPQPTKKASGMLSFFRGTAGKSPDLSSQKRETLRGADSAYYQVGQTGKEGTENQGVEPQDEVDGGDTQKKQLINPHVELQFSDANAKFYCRLYYAGEFHKMREVILDSSEEDFIRSLSHSSPWQARGGKSGAAFYATEDDRFILKQMPRLEVQSFLDFAPHYFNYITNAVQQKRPTALAKILGVYRIGYKNSQNNTEKKLDLLVMENLFYGRKMAQVFDLKGSLRNRNVKTDTGKESCDVVLLDENLLKMVRDNPLYIRSHSKAVLRTSIHSDSHFLSSHLIIDYSLLVGRDDTSNELVVGIIDYIRTFTWDKKLEMVVKSTGILGGQGKMPTVVSPELYRTRFCEAMDKYFLMVPDHWTGLGLNC

SEQ ID NO: 3 - DNA, 6159 bp, Mus musculus
  CDS (1)..(6159)
atggccacagatgacaagagttccccgacactggactctgctaatgatttgcctcgctctcctgccagtccttctcacctcactcactttaaacccttgactcctgaccaggatgagccccccttcaagtcagcatatagttcttttgtaaacctttttcgttttaacaaagagcgaggagaagggggccaaggagagcagcagtctccgagttcaagttgggccagccctcagatcccttcaagaacacagtctgtgaggtcgcctgtaccttataaaaaacagcttaatgaggagctccaccggcgctcttcagtgttagagaacactttgccacatcctcaggagagcacagactccagaaggaaagcagaaccagcctgtggaggtcatgacccacgtacagctgttcagcttcgaagcctcagcacagtattgaaacgcctcaaagaaatcatggaaggaaaaagccaggacagtgacctgaagcaatattggatgccagatagccagtgtaaagagtgctatgactgcagtgaaaagtttacaacatttaggcgcagacaccattgcagactgtgtgggcagattttctgcagtcgttgttgtaatcaagaaatccctggaaaatttatgggctatacaggagacctccgagcatgcacctactgtagaaaaatagccttaagttatgctcattctacagacagtaattccattggggaagacttgaatgctctttcagattcaacttgctctgtgtctatacttgatccaagcgaacctcggacaccagttgggagtagaaaagccagtcgtaacatattcttagaggatgatttagcctggcaaagcttgattcatccggattcctcaaatagtgctctttcaacaagactcgtatctgttcaagaggatgctgggaagtctcctgctcgaaacagatcagccagcattactaatctgtcactggatcggtctggttctcctatggttccttcatatgagacatctgtcagtccccaggctaaccgaaactacattaggacagagacgactgaggatgaacgcaaaattcttctggacagtgctcagttaaaggatctgtggaagaaaatctgccatcacaccagtgggatggaatttcaagatcaccgttactggttgagaacacatcccaactgcattgtagggaaggaattagtcaactggctaatcagaaatggacacatcgctacaagggcacaagctatagcaattggacaagcaatggttgatggacgttggttggattgtgttagtcatcatgatcagcttttcagggacgaatatgcgttgtatagaccacttcagagtacagaattttctgagacaccttctccagacagtgactctgtgaactctgtggaaggacactccgagccatcctggtttaaagacataaaatttgatgacagtgacacagaacagattgctgaagaaggtgacgataatttggctaagtatttggtttctgacactggaggacagcagctctcaataagtgatgccttcatcaaagagtccttatttaatcgacgagtagaggaaaaatccaaagagctgccttttacccctttgggctggcatcataacaacctggaactcttgcgagaggagaatgaggagaagcaagccatggaaaggctgctttcagctaatcataaccacatgatggccctactccagcagttgcttcaaaacgagtcattgtcatcgtcttggagggacatcattgtgtcactagtctgccaggttgttcagacagtccgacctgatgtcaagcaccaggatgatgacatggatatccgtcagtttgtccatatcaagaagatcccaggtggaaagaaatttgactctgtggttgtcaatggctttgtttgtaccaagaacattgcacataaaaagatgaattcctgtattaaaaaccccaaaatccttctgttgaagtgttctattgagtatctctatagagaagaaactaagtttacctgcattgatcctattgtgcttcaggaaagggaattcttgaagaattatgttcaacgaatagttgatgttcgacccacattggttcttgttgagaaaacagtgtctcggattgctcaggacatgttactggaacatggcattactctggtcattaatgtaaagtcacaagttttagaaagaatcagtcgaatgacccaaggtgatttagtggtgtccatggaccagctgctcaccaaaccccacttgggcacttgccacaaattttatatgcagatatttcagctgcctaatgaacaaaccaaaacactgatgttttttgaaggttgtccacagcatctaggctgcacaatcaagctcagaggaggctctgattatgagctggctcgagttaaggagatcctaatatttatgatctgtgtagcttatcattctcagctagaaatctcttttctcatggatgagttcgctatgcctccaacattaatgcaaagcccttcattccatcttctgacggagggacgaggtgaagagggagcctctcaggagcaggtcagtggcagctcccttcctcaggatcctgagtgccctcgtgaggccctgtcttctgaggatagcactttgttggaatcaaggactgtgctagagaagggtgaactagacaataaaagtattccacaagctgttgcctctttgaagcatcaagattataccacccccacttgcccagcaggtattccctgtgctctttttgcattggtaccagagtcattgttgcctctccatatggatcaacaggatgccgtaggaaatgaacaccgagagacttcacagcaaacggatgagcaacaggatcccaaaagccagatgaaagcttttagagaccctttacaggatgacactggaatgtacgttactgaggaagtcacctcctctgaagatcaacgaaagacttatgccttgacatttaaacaggagttaaaagatgtaatcctctgtatctctccagttattacattccgtgaacctttccttttaactgaaaaggggatgagatgctcaactcgagattattttccagagcagatttactggtctcctcttctcaacaaagaggtgaaggaaatggagagcaggaggaagaaacagctgctcagggatctctctggacttcagggcatgaatggcagtgttcaggccaagtctattcaagtcttaccctcacatgagctagtgagcaccaggattgctgaacatgtgggtgacagccagaccttgggtagaatgctagctgattatcgagctagaggaggagaattcagtcaaaacatttggaacccctttgtccattcaaaagatgacatcatgtacttcaggtggcaaatcagggaaacaaaactgagagtgatgaaagagaggggattgattccaagtgatgtaatatggccaacaaaggtggactgtctgaaccctgctaaccaccagaggctctgtgtgctcttcagcagctcttctgcccagtccagcaatgctcccagtgcttgtgtcagtccttggattgtaacaatggagttttatggaaagaatgaccttacactggggatatttttagaaagatactgtttcaggtactcttaccagtgtccgagcatgttctgtgacacccccatggttcatcacattcgacgctttgttcatggccaaggctgtgtacagataattctgaaggagttggattctccagtgcctggatatcaacatacaattctcacatattcctggtgcagaatctgcaaacaagtaacaccagttgttgctctttcaaatgaatcctggtctatgtcatttgcaaagtaccttgaacttcgattttatggccaccagtacacacgcagagccaacgctgagccctgcggtcactctatccaccatgattatcaccagtatttctcttataaccagatggtggcatctttcagttactctcctattcggcttcttgaagtatgtgttccactaccaaaaatattcattaagcgtcaagccccactgaaggtatctcttcttcaggacctcaaagacttttttcagaaggtttcacaggtgtacctagctgttgatgagagacttgcatccttgaaaacggatacatttagcaaaactagagaggaaaagatggaagatatctttgcacaaaaggagatggaagagggtgagtttaagaactggacagagaagatgcaagcaaggctcatgtcttcctctgtggatacccctcagcaactgcagtccatttttgagtcactgattgccaagaagcaaagcctctgtgaggtgctccaggcgtggaacagcaggttgcaggacctcttccagcaggaaaaaggtagaaagaggccttcagttcctcccagtcctgggagactgagacaaggtgaagaaagcaagataaatgcaatggacacatctccaaggaatatttctccaggactttcacaatggagaaaaagaagatcgcttcttgacaaccctgtccagccagctacgagctccacccacctccagctgcccactcctcccgaggccctggccgagcaggtagtgggagggccgactgatctggattcagccagtggctctgaagatgtatttgatggtcatttgctgggatccacagacagccaggtgaaggaaaagtcaaccatgaaagccatctttgctaatttgcttccaggaaacagctacaatcccattccatttccttttgatccagataaacactacttaatgtatgaacatgaacgggtgcccattgctgtctgtgagaaagagcccagctccatcattgcttttgcactcagttgtaaagaataccgcaatgccttagaggaattgtccaaagcaactctgcggaacagtgctgaagaagggctcccagccaatagtgctttagataacagacctaagagtagcagccctattagactacctgaaatcagtggaggacagacaaaccgcacagtagaagcagaacctcagccaaccaaaaaagcttcaggaatgttgtccttcttcagaggaacagcagggaagagccctgatctgtcttcccagaagagggagaccttgcgaggggcagacagtgcttactaccaggttgggcaggccggcaaggaggggttggagagtcaaggcctggagcctcaagatgaagtagatggaggagatacacagaagaaacaactcacaaatcctcatgtggaacttcaattttctgatgctaatgccaagttttactgtcggctgtactacgcgggagagttccacaagatgcgtgaagtgattctgggcagcagtgaggaggaattcatccgttccctttctcactcatctccctggcaggcccggggaggcaagtcaggagctgctttctatgccaccgaagatgatagattcattctgaagcaaatgcctcgtttggaagtccagtctttccttgactttgcaccacactacttcaattatatcacaaatgctgttcaacaaaagaggcccaccgccttggctaaaattcttggagtttacagaattggttataagaactctcagaacaacactgagaagaagttagatctccttgtcatggaaaatcttttctatgggagaaagatggcacaggtttttgatttgaagggttcacttaggaatcgaaatgtaaaaactgacactgggaaagagagctgtgatgtggttctgttggatgaaaacctcctaaagatggttcgagacaaccctctctatattcgttcccattccaaatctgagctgagaacctccatccacagcgacgcccatttcctttccagccacctcattatagactattctctgctggttgggcgagatgacactagcaatgagcttgtggttggcatcatagattacattcgaacatttacatgggacaaaaaacttgagatggttgtgaagtcaacaggaattttaggaggacaaggtaaaatgccaactgtggtctctccagagttgtataggactagattttgtgaagcaatggacaagtatttcttgatggtgccagaccactggacagggttggatctgaattgctga

SEQ ID NO: 4 - protein, 2052 aa, Mus musculus
MATDDKSSPTLDSANDLPRSPASPSHLTHFKPLTPDQDEPPFKSAYSSFVNLFRFNKERGEGGQGEQQSPSSSWASPQIPSRTQSVRSPVPYKKQLNEELHRRSSVLENTLPHPQESTDSRRKAEPACGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSTCSVSILDPSEPRTPVGSRKASRNIFLEDDLAWQSLIHPDSSNSALSTRLVSVQEDAGKSPARNRSASITNLSLDRSGSPMVPSYETSVSPQANRNYIRTETTEDERKILLDSAQLKDLWKKICHHTSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLAKYLVSDTGGQQLSISDAFIKESLFNRRVEEKSKELPFTPLGWHHNNLELLREENEEKQAMERLLSANHNHMMALLQQLLQNESLSSSWRDIIVSLVCQVVQTVRPDVKHQDDDMDIRQFVHIKKIPGGKKFDSVVVNGFVCTKNIAHKKMNSCIKNPKILLLKCSIEYLYREETKFTCIDPIVLQEREFLKNYVQRIVDVRPTLVLVEKTVSRIAQDMLLEHGITLVINVKSQVLERISRMTQGDLVVSMDQLLTKPHLGTCHKFYMQIFQLPNEQTKTLMFFEGCPQHLGCTIKLRGGSDYELARVKEILIFMICVAYHSQLEISFLMDEFAMPPTLMQSPSFHLLTEGRGEEGASQEQVSGSSLPQDPECPREALSSEDSTLLESRTVLEKGELDNKSIPQAVASLKHQDYTTPTCPAGIPCALFALVPESLLPLHMDQQDAVGNEHRETSQQTDEQQDPKSQMKAFRDPLQDDTGMYVTEEVTSSEDQRKTYALTFKQELKDVILCISPVITFREPFLLTEKGMRCSTRDYFPEQIYWSPLLNKEVKEMESRRKKQLLRDLSGLQGMNGSVQAKSIQVLPSHELVSTRIAEHVGDSQTLGRMLADYRARGGEFSQNIWNPFVHSKDDIMYFRWQIRETKLRVMKERGLIPSDVIWPTKVDCLNPANHQRLCVLFSSSSAQSSNAPSACVSPWIVTMEFYGKNDLTLGIFLERYCFRYSYQCPSMFCDTPMVHHIRRFVHGQGCVQIILKELDSPVPGYQHTILTYSWCRICKQVTPVVALSNESWSMSFAKYLELRFYGHQYTRRANAEPCGHSIHHDYHQYFSYNQMVASFSYSPIRLLEVCVPLPKIFIKRQAPLKVSLLQDLKDFFQKVSQVYLAVDERLASLKTDTFSKTREEKMEDIFAQKEMEEGEFKNWTEKMQARLMSSSVDTPQQLQSIFESLIAKKQSLCEVLQAWNSRLQDLFQQEKGRKRPSVPPSPGRLRQGEESKINAMDTSPRNISPGLSQWRKRRSLLDNPVQPATSSTHLQLPTPPEALAEQVVGGPTDLDSASGSEDVFDGHLLGSTDSQVKEKSTMKAIFANLLPGNSYNPIPFPFDPDKHYLMYEHERVPIAVCEKEPSSIIAFALSCKEYRNALEELSKATLRNSAEEGLPANSALDNRPKSSSPIRLPEISGGQTNRTVEAEPQPTKKASGMLSFFRGTAGKSPDLSSQKRETLRGADSAYYQVGQAGKEGLESQGLEPQDEVDGGDTQKKQLTNPHVELQFSDANAKFYCRLYYAGEFHKMREVILGSSEEEFIRSLSHSSPWQARGGKSGAAFYATEDDRFILKQMPRLEVQSFLDFAPHYFNYITNAVQQKRPTALAKILGVYRIGYKNSQNNTEKKLDLLVMENLFYGRKMAQVFDLKGSLRNRNVKTDTGKESCDVVLLDENLLKMVRDNPLYIRSHSKSELRTSIHSDAHFLSSHLIIDYSLLVGRDDTSNELVVGIIDYIRTFTWDKKLEMVVKSTGILGGQGKMPTVVSPELYRTRFCEAMDKYFLMVPDHWTGLDLNC

SEQ ID NO: 5 - DNA, 2349 bp, Homo sapiens
  CDS (1)..(2349)
atgaaccccgagaaggatttcgcgccgctcacgcctaacatcgtgcgcgccctcaatgacaagctgtacgaaaagcggaaggtggcagcgctggagatcgagaagctggtccgggagttcgtggcccagaacaataccgtgcaaatcaagcatgtgatccagaccctgtcccaggagtttgccctgtctcagcacccccacagccggaaagggggcctcatcggcctggccgcctgctccatcgcactgggcaaggactcagggctctacctgaaggagctgatcgagccagtgctgacctgcttcaatgatgcagacagcaggctgcgctactatgcctgcgaggccctctacaacatcgtcaaggtggcccggggcgctgtgctgccccacttcaacgtgctctttgacgggctgagcaagctggcagccgacccagaccccaatgtgaaaagcggatctgagctcctagaccgccttttaaaggacattgtgactgagagcaacaagtttgacctggtgagcttcatccccttgttgcgagagaggatttactccaacaaccagtatgcccggcagttcatcatctcctggatcctggttctggagtcggtgccagacattaacctgctggattacctgccggagatcctggatggactcttccagatcctgggtgacaatggcaaagagattcgcaaaatgtgtgaggttgttcttggagaattcttaaaagaaattaagaagaacccctccagtgtgaagtttgctgagatggccaacatcctggtgatccactgccagacaacagatgacctcatccagctgacagccatgtgctggatgcgggagttcatccagctggcgggccgcgtcatgctgccttactcctccgggatcctgactgctgtcttgccctgcttggcctacgatgaccgcaagaaaagcatcaaagaagtggccaacgtgtgcaaccagagcctgatgaagctggtcacccccgaggacgacgagctggatgagctgagacctgggcagaggcaggcagagcccacccctgacgatgccctgccaaagcaggagggcacagccagtggaggtccagatggttcctgtgactccagcttcagtagcggcatcagtgtcttcactgcagccagcactgaaagagccccagtgacccttcacctcgacgggatcgtgcaggtcctaaactgccacctcagtgacacggccattgggatgatgaccaggattgcagttctcaagtggctctaccacctctacatcaaaactcctcggaagatgttccggcacacggacagcctctttcccatcctactgcagacgttatcggatgaatcggatgaggtgatcctgaaggacctggaggtgctggcagaaatcgcttcctcccccgcaggccagacggatgacccaggccccctcgatggccctgacctccaggccagccactcagagctccaggtgcccacccctggcagagccggcctactgaacacctctggtaccaaaggcttagaatgttctccttcaactcccaccatgaattcttacttttataagttcatgatcaaccttctcaagagattcagcagcgaacggaagctcctggaggtcagaggccctttcatcatcaggcagctgtgcctcctgctgaatgcggagaacatcttccactcaatggcagacatcctgctgcgggaggaggacctcaagttcgcctcgaccatggtccacgccctcaacaccatcctgctgacctccacagagctcttccagctaaggaaccagctgaaggacctgaagaccctggagagccagaacctgttctgctgcctgtaccgctcctggtgccacaacccagtcaccacggtgtccctctgcttcctcacccagaactaccggcacgcctatgacctcatccagaagtttggggacctggaggtcaccgtggacttcctcgcagaggtggacaagctggtgcagctgattgagtgccccatcttcacatatctgcgcctgcagctgctggacgtgaagaacaacccctacctgatcaaggccctctacggcctgctcatgctcctgccgcagagcagcgccttccagctgctctcgcaccggctccagtgcgtgcccaaccctgagctgctgcagaccgaagacagtctaaaggcagcccccaagtcccagaaagctgactcccctagcatcgactacgcagagctgctgcagcactttgagaaggtccagaacaagcacctggaagtgcggcaccagcggagcgggcgtggggaccacctggaccggagggttgtcctctga

SEQ ID NO: 6 - protein, 782 aa, Homo sapiens
MNPEKDFAPLTPNIVRALNDKLYEKRKVAALEIEKLVREFVAQNNTVQIKHVIQTLSQEFALSQHPHSRKGGLIGLAACSIALGKDSGLYLKELIEPVLTCFNDADSRLRYYACEALYNIVKVARGAVLPHFNVLFDGLSKLAADPDPNVKSGSELLDRLLKDIVTESNKFDLVSFIPLLRERIYSNNQYARQFIISWILVLESVPDINLLDYLPEILDGLFQILGDNGKEIRKMCEVVLGEFLKEIKKNPSSVKFAEMANILVIHCQTTDDLIQLTAMCWMREFIQLAGRVMLPYSSGILTAVLPCLAYDDRKKSIKEVANVCNQSLMKLVTPEDDELDELRPGQRQAEPTPDDALPKQEGTASGGPDGSCDSSFSSGISVFTAASTERAPVTLHLDGIVQVLNCHLSDTAIGMMTRIAVLKWLYHLYIKTPRKMFRHTDSLFPILLQTLSDESDEVILKDLEVLAEIASSPAGQTDDPGPLDGPDLQASHSELQVPTPGRAGLLNTSGTKGLECSPSTPTMNSYFYKFMINLLKRFSSERKLLEVRGPFIIRQLCLLLNAENIFHSMADILLREEDLKFASTMVHALNTILLTSTELFQLRNQLKDLKTLESQNLFCCLYRSWCHNPVTTVSLCFLTQNYRHAYDLIQKFGDLEVTVDFLAEVDKLVQLIECPIFTYLRLQLLDVKNNPYLIKALYGLLMLLPQSSAFQLLSHRLQCVPNPELLQTEDSLKAAPKSQKADSPSIDYAELLQHFEKVQNKHLEVRHQRSGRGDHLDRRVVL

SEQ ID NO: 7 - DNA, 2349 bp, Mus musculus
  CDS (1)..(2349)
atgaacccagagaaggattttgcgccgctcactcccaacatcgtaagggccctgaatgataaactctacgaaaagcggaaggttgcagcgctggagatcgagaagctggtgcgggactttgtggcccagaacaacaccatgcaaatcaagcacgtgatccagaccctgtctcaggagtttgccctgtcccagcacccccacagccggaaagggggcctcatcggtctggcagcctgctctattgccctaggcaaggactcagggctctacctgaaggagctgatagagccagtgctgacctgctttaatgacgcagacagcaggctgcgctactacgcttgtgaggccctctacaatatcgtcaaagtggcccgaggtgccgtgctgccgcacttcaatgttctcttcgatggactgagtaagttggctgctgacccagaccccaatgtgaaaagtggatctgagctcctggaccgcctcttaaaggacattgtgacagaaagcagcaagtttgacctggtgagcttcatcccccttttgcgggagaggatttactccaacaaccaatacgcccgacagttcatcatctcctggatcctggttctggtgtcggtgccagacattaacctgctggattacctgccagagatcctagatgggctcttccagatcctgggtgacaatggcaaggagattcggaaaatgtgtgaggtggttcttggagagttcttgaaagaaattaagaagaacccatccagtgtgaagtttgctgagatggccaacatcctggtgatccactgccagaccacagatgacctgattcagctgacggctatgtgctggatgcgggaattcatccagctggcgggccgggtaatgctgccctattcctctgggatcctgactgctgtcctgccatgcttggcctatgatgaccgcaagaaaagtatcaaggaggtggccaatgtgtgtaaccagagcttgatgaagctggtcacccctgaggatgatgaacccgatgagccaaagtcagtagcacagaagcagaccgaacccaaccctgaggattccctgccaaagcaggaggggacagccagtggaggtccaggttcctgtgactccagctttggaagtggcatcaatgtcttcacctcagccaacactgatagggctccagtgaccctgcacctggacgggattgttcaagtcctgaactgtcaccttagtgacacaaccattgggatgatgaccaggattgctgttctgaagtggctctaccatctctacatcaaaactcctcgaaagatgttccggcacacagacagcctcttccccatcttacttcagacattatctgacgaatctgatgaggttgtcttgaaggatctggaagtgttggcagaaattgcttcctcccctgcaggccagacggatgacccaggtgcacctgatggccctgacctccgagtcaaccactcggagcttcaggtgcccacctccggcagagccaacctactaaaccctcccagtaccaaaggcttagaaggctccccctccactcccaccatgaactcctacttttacaagttcatgatcaaccttctacagacgttcagcagtgaacggaagctcctggaggccagaggcccgttcatcatcaggcagctctgcctcctgctgaatgcagagaacatcttccattcaatggcagacatcctgcttcgggaggaggacctcaagttcgcgtccaccatggtgcacaccctcaacaccatcctgctcacctccaccgagctcttccagctgaggaaccagctcaaggacctgcagaccccggaaagccagaacctcttctgctgcctgtaccgctcctggtgccacaacccagtcaccaccgtgtccctctgcttcctcacccagaactaccgccacgcctatgacctcatccagaagtttggggacctggaagtcacagtggatttcctcacagaggtggacaaactcgtgcagctaattgagtgccccatctttacatatctacgcctgcaactgctggacgtgaagaacaacccatacctgatcaaggccctgtacggcctgctcatgctgttgccccagagcagtgccttccagctgctctctcaccggctccagtgtgtgcccaaccctgagctgctgcagactgaagactgtctgaaagcggcccccaagtcccagaaaggtgactcccccagcatcgactacacagagctgctgcagcactttgaaaaagtccagaaacagcacctggaagtaagacaccagcgcagtgggcgaggggatcacctggaccgcagagttatcctctga

SEQ ID NO: 8 - protein, 782 aa, Mus musculus
MNPEKDFAPLTPNIVRALNDKLYEKRKVAALEIEKLVRDFVAQNNTMQIKHVIQTLSQEFALSQHPHSRKGGLIGLAACSIALGKDSGLYLKELIEPVLTCFNDADSRLRYYACEALYNIVKVARGAVLPHFNVLFDGLSKLAADPDPNVKSGSELLDRLLKDIVTESSKFDLVSFIPLLRERIYSNNQYARQFIISWILVLVSVPDINLLDYLPEILDGLFQILGDNGKEIRKMCEVVLGEFLKEIKKNPSSVKFAEMANILVIHCQTTDDLIQLTAMCWMREFIQLAGRVMLPYSSGILTAVLPCLAYDDRKKSIKEVANVCNQSLMKLVTPEDDEPDEPKSVAQKQTEPNPEDSLPKQEGTASGGPGSCDSSFGSGINVFTSANTDRAPVTLHLDGIVQVLNCHLSDTTIGMMTRIAVLKWLYHLYIKTPRKMFRHTDSLFPILLQTLSDESDEVVLKDLEVLAEIASSPAGQTDDPGAPDGPDLRVNHSELQVPTSGRANLLNPPSTKGLEGSPSTPTMNSYFYKFMINLLQTFSSERKLLEARGPFIIRQLCLLLNAENIFHSMADILLREEDLKFASTMVHTLNTILLTSTELFQLRNQLKDLQTPESQNLFCCLYRSWCHNPVTTVSLCFLTQNYRHAYDLIQKFGDLEVTVDFLTEVDKLVQLIECPIFTYLRLQLLDVKNNPYLIKALYGLLMLLPQSSAFQLLSHRLQCVPNPELLQTEDCLKAAPKSQKGDSPSIDYTELLQHFEKVQKQHLEVRHQRSGRGDHLDRRVIL

SEQ ID NO: 9 - DNA, 966 bp, Homo sapiens
  CDS (1)..(966)
atgaagcaactgccagtcttggaacctggagacaagcccaggaaagcaacatggtacaccttgactgtccctggagacagcccctgtgctcgagttggccacagctgttcatatttacccccagttggtaatgccaagagagggaaggtcttcattgttgggggagcaaatccaaacagaagcttctcagacgtgcacaccatggatctggaaaccaggacgtggaccacgccagaagtgaccagccccccaccatccccaagaacattccacacatcatcggcagccattggaaaccagctatatgtctttgggggcggagagagaggtgcccagcccgtgcaggacacgaagctgcatgtgtttgacgcaaacactctgacctggtcacagccagagacacttggaaatcctccatctccccggcatggtcatgtgatggtggcagcagggacaaagctcttcatccacggaggcttggcgggggacagattctatgatgacctccactgcattgatataagtgacatgaaatggcagaagctaaatcccactggggctgctccagcaggctgtgctgcccactcagctgtggccatgggaaaacatgtgtacatctttggtggaatgactcctgcaggagcactggacacaatgtaccagtatcacacagaagagcagcattggaccttgcttaaatttgatactcttctaccccctggacgattggaccattccatgtgtatcattccatggccagtgacgtgtgcttctgagaaagaagattccaactctctcactctgaaccatgaagctgagaaagaggattcagctgacaaagtaatgagccacagtggtgactcacatgaggaaagccagactgctacactgctctgtttggtgtttggtgggatgaatacagaaggggaaatctatgacgattgtattgtgactgtagtggactaa

SEQ ID NO: 10 - protein, 321 aa, Homo sapiens
MKQLPVLEPGDKPRKATWYTLTVPGDSPCARVGHSCSYLPPVGNAKRGKVFIVGGANPNRSFSDVHTMDLETRTWTTPEVTSPPPSPRTFHTSSAAIGNQLYVFGGGERGAQPVQDTKLHVFDANTLTWSQPETLGNPPSPRHGHVMVAAGTKLFIHGGLAGDRFYDDLHCIDISDMKWQKLNPTGAAPAGCAAHSAVAMGKHVYIFGGMTPAGALDTMYQYHTEEQHWTLLKFDTLLPPGRLDHSMCIIPWPVTCASEKEDSNSLTLNHEAEKEDSADKVMSHSGDSHEESQTATLLCLVFGGMNTEGEIYDDCIVTVVD

SEQ ID NO: 11 - DNA, 21 bp, Artificial Sequence
  Vac14 siRNA (ID# s107929) sense
gauuuacuccaacaaccaatt

SEQ ID NO: 12 - DNA, 21 bp, Artificial Sequence
  Vac14 siRNA (ID# s107929) antisense
uugguuguuggaguaaaucct

SEQ ID NO: 13 - DNA, 21 bp, Artificial Sequence
  PIKfyve siRNA sense
gacguccccaacacuggactt

SEQ ID NO: 14 - DNA, 21 bp, Artificial Sequence
  PIKfyve siRNA antisense
guccaguguuggggacguctt

SEQ ID NO: 15 - DNA, 21 bp, Artificial Sequence
  PIKfyve siRNA sense
cacuggacucugcuaaugatt

SEQ ID NO: 16 - DNA, 21 bp, Artificial Sequence
  PIKfyve siRNA antisense
ucauuagcagaguccagugtt

SEQ ID NO: 17 - DNA, 21 bp, Artificial Sequence
  PIKfyve siRNA sense
ugauuugccucgaucuccutt

SEQ ID NO: 18 - DNA, 21 bp, Artificial Sequence
  PIKfyve siRNA antisense
aggagaucgaggcaaaucatt

SEQ ID NO: 19 - DNA, 21 bp, Artificial Sequence
  PIKfyve siRNA sense
cagcagccuuugaguggaatt

SEQ ID NO: 20 - DNA, 21 bp, Artificial Sequence
  PIKfyve siRNA antisense
uuccacucaaaggcugcugtt

SEQ ID NO: 21 - DNA, 21 bp, Artificial Sequence
  PIKfyve siRNA sense
aaagcagcuuaaugaggaatt

SEQ ID NO: 22 - DNA, 21 bp, Artificial Sequence
  PIKfyve siRNA antisense
uuccucauuaagcugcuuutt

SEQ ID NO: 23 - DNA, 21 bp, Artificial Sequence
  PIKfyve siRNA sense
ggaaagcagaaccuaccuutt

SEQ ID NO: 24 - DNA, 21 bp, Artificial Sequence
  PIKfyve siRNA antisense
aagguagguucugcuuucctt

SEQ ID NO: 25 - DNA, 21 bp, Artificial Sequence
  PIKfyve siRNA sense
ccuaccuuuggaggucaugtt

SEQ ID NO: 26 - DNA, 21 bp, Artificial Sequence
  PIKfyve siRNA antisense
caugaccuccaaagguaggtt

SEQ ID NO: 27 - DNA, 21 bp, Artificial Sequence
  PIKfyve siRNA sense
cgccucaaggaaaucauggtt

SEQ ID NO: 28 - DNA, 21 bp, Artificial Sequence
  PIKfyve siRNA antisense
ccaugauuuccuugaggcgtt

SEQ ID NO: 29 - DNA, 21 bp, Artificial Sequence
  PIKfyve siRNA sense
guuaugcucauuccacagatt

SEQ ID NO: 30 - DNA, 21 bp, Artificial Sequence
  PIKfyve siRNA antisense
ucuguggaaugagcauaactt

SEQ ID NO: 31 - DNA, 21 bp, Artificial Sequence
  PIKfyve siRNA sense
cauauguuaggacagagactt

SEQ ID NO: 32 - DNA, 21 bp, Artificial Sequence
  PIKfyve siRNA antisense
gucucuguccuaacauaugtt

SEQ ID NO: 33 - DNA, 21 bp, Artificial Sequence
  vac14 siRNA sense
ccccgagaaggauuucgcgtt

SEQ ID NO: 34 - DNA, 21 bp, Artificial Sequence
  vac14 siRNA antisense
cgcgaaauccuucucggggtt

SEQ ID NO: 35 - DNA, 21 bp, Artificial Sequence
  vac14 siRNA sense
aagcggaagguggcagcgctt

SEQ ID NO: 36 - DNA, 21 bp, Artificial Sequence
  vac14 siRNA antisense
gcgcugccaccuuccgcuutt

SEQ ID NO: 37 - DNA, 21 bp, Artificial Sequence
  vac14 siRNA sense
aucaagcaugugauccagatt

SEQ ID NO: 38 - DNA, 21 bp, Artificial Sequence
  vac14 siRNA antisense
ucuggaucacaugcuugautt

SEQ ID NO: 39 - DNA, 21 bp, Artificial Sequence
  vac14 siRNA sense
ggagcugaucgagccagugtt

SEQ ID NO: 40 - DNA, 21 bp, Artificial Sequence
  vac14 siRNA antisense
cacuggcucgaucagcucctt

SEQ ID NO: 41 - DNA, 21 bp, Artificial Sequence
  vac14 siRNA sense
gcggaucugagcuccuagatt

SEQ ID NO: 42 - DNA, 21 bp, Artificial Sequence
  vac14 siRNA antisense
ucuaggagcucagauccgctt

SEQ ID NO: 43 - DNA, 21 bp, Artificial Sequence
  vac14 siRNA sense
guuugaccuggugagcuuctt

SEQ ID NO: 44 - DNA, 21 bp, Artificial Sequence
  vac14 siRNA antisense
gaagcucaccaggucaaactt

SEQ ID NO: 45 - DNA, 21 bp, Artificial Sequence
  vac14 siRNA sense
auuaagaagaaccccuccatt

SEQ ID NO: 46 - DNA, 21 bp, Artificial Sequence
  vac14 siRNA antisense
uggagggguucuucuuaautt

SEQ ID NO: 47 - DNA, 21 bp, Artificial Sequence
  vac14 siRNA sense
guuugcugagauggccaactt

SEQ ID NO: 48 - DNA, 21 bp, Artificial Sequence
  vac14 siRNA antisense
guuggccaucucagcaaactt

SEQ ID NO: 49 - DNA, 21 bp, Artificial Sequence
  vac14 siRNA sense
aagcaucaaagaaguggcctt

SEQ ID NO: 50 - DNA, 21 bp, Artificial Sequence
  vac14 siRNA antisense
ggccacuucuuugaugcuutt

SEQ ID NO: 51 - DNA, 21 bp, Artificial Sequence
  vac14 siRNA sense
gcuccuggaggucagaggctt

SEQ ID NO: 52 - DNA, 21 bp, Artificial Sequence
  vac14 siRNA antisense
gccucugaccuccaggagctt

SEQ ID NO: 53 - DNA, 20 bp, Artificial Sequence
  DAPDH primer forward
atcactgccacccagaagac

SEQ ID NO: 54 - DNA, 20 bp, Artificial Sequence
  DAPDH primer reverse
ggatgcagggatgatgttct

SEQ ID NO: 55 - DNA, 20 bp, Artificial Sequence
  IL12b primer forward
cagcaccagcttcttcatca

SEQ ID NO: 56 - DNA, 20 bp, Artificial Sequence
  IL12b primer reverse
tactcccagctgacctccac